(19) United States
(12) Patent Application Publication
(10) Pub. No.: US 2002/0150906 A1
(43) Pub. Date: Oct. 17, 2002

(54) Method for determining three-dimensional protein structure from primary protein sequence

(75) Inventor: Derek A. Debe, Pasadena, CA (US)

Correspondence Address: LYON & LYON LLP, 633 WEST FIFTH STREET, SUITE 4700, LOS ANGELES, CA, 90071, US

(73) Assignee: California Institute of Technology, Pasadena, CA

(21) Appl. No.: 09/905,176

(22) Filed: Jul. 12, 2001

Related U.S. Application Data
(60) Provisional application No. 60/218,016, filed on Jul. 12, 2000.

(51) Int. Cl. C12Q 1/68; G06F 19/00; G01N 33/48; G01N 33/50

(52) U.S. Cl. 435/6; 702/20

(57) ABSTRACT

A preferred embodiment of the invention is a method for determining a preferred sequence alignment between a query sequence and one or more template sequences comprising the steps of: 1) aligning at least two reference sequences to determine one or more BRIDGE/BULGE gaps; 2) determining an alignment score between each potential alignment of the query sequence and each template sequence based on whether or not a given sequence alignment between the query sequence and each template sequence creates a BRIDGE/BULGE gap and 3) determining a preferred sequence alignment based on the alignment scores of the query sequence with each template sequence.

### FIELD OF THE INVENTION

The invention relates to the field of computational methods for determining protein homology relationships.

### BACKGROUND

While the sequencing of the human genome is a landmark achievement in genomics, it also creates the next great challenge, namely to create an accurate structural model of each protein coded by the human genome. Since the experimental determination of all of the protein structures coded would require decades, computational methods for determining three-dimensional protein structures are essential if structural genomics is going to rapidly progress. S. K. Burley, S. C. Almo, J. B. Bonanno et al., Nature Gen.23,151-157 (1999). This reference and all other references cited herein are incorporated by reference.

Proteins are linear polymers of amino acids. Naturally occurring proteins may contain as many as 20 different types of amino acid residues, each of which contains a distinctive side chain. The particular linear sequence of amino acid residues in a protein define the primary sequence, or primary structure, of the protein. The primary structure of a protein can be determined with relative ease using known methods.

Proteins fold into a three-dimensional structure. The folding is determined by the sequence of amino acids and by the protein's environment. Examination of the three-dimensional structure of numerous natural proteins has revealed a number of recurring patterns. Patterns known as alpha helices, parallel beta sheets, and anti-parallel beta sheets are commonly observed. A description of these common structural patterns is provided by Dickerson, R. E., et al. in The Structure and Action of Proteins, W. A. Benjamin, Inc. California (1969). The assignment of each amino acid residue to one of these patterns defines the secondary structure of the protein.

The biological properties of a protein depend directly on its three-dimensional (3D) conformation. The 3D conformation determines the activity of enzymes, the capacity and specificity of binding proteins, and the structural attributes of receptor molecules. Because the three-dimensional structure of a protein molecule is so significant, it has long been recognized that a means for easily determining a protein's three-dimensional structure from its known amino acid sequence would be highly desirable. However, it has proven extremely difficult to make such a determination without experimental data.

In the past, the three-dimensional structures of proteins have been determined using a number of different experimental methods. Perhaps the recognized methods of determining protein structure involves the use of the technique of x-ray crystallography. A general review of this technique can be found in Physical Bio-chemistry, Van Holde, K. E. (Prentice-Hall, New Jersey 1971), pp. 221-239, or in Physical Chemistry with Applications to the Life Sciences, D. Eisenberg & D. C. Crothers (Benjamin Cummings, Menlo Park 1979). Using this technique, it is possible to elucidate three-dimensional structure with precision. Additionally, protein structure may be determined through the use of neutron diffraction techniques, or by nuclear magnetic resonance (NMR). See, e.g., Physical Chemistry, 4th Ed. Moore, W. J. (Prentice-Hall, New Jersey 1972) and NMR of Proteins and Nucleic Acids, K. Wuthrich (Wiley-Interscience, New York 1986).

These experimental techniques all suffer from at least one significant shortcoming. Namely, they are labor intensive and therefore slow and expensive. Modern sequencing techniques are creating rapidly growing databases of primary sequences that need to be translated into three dimensional protein structures. Indeed, with more than 500 genomes including the human genome fully sequenced, three dimensional structures have only been determined for about 2% of these sequences. Every day the ratio of predicted-three dimensional structures to primary sequences is getting smaller.

In order to more rapidly predict three dimensional structures from primary sequences, biochemists are turning to various computational approaches that permit structure determination to be done with computers and software rather than laborious and intricate laboratory techniques. One of the most promising of these computational approaches compares the similarity of a primary sequence for which the three dimensional structure of the sequence is sought, referred to throughout as a query sequence or a query peptide against one or more primary sequences, usually a database of such sequences, referred to throughout as template sequences or template peptides, for which the three dimensional structures are known. This is one aspect of primary sequence homology modeling.

At a high level, many primary sequence homology modeling methods can be characterized in two steps. In the first step, referred to as the alignment step, the query sequence for which the three dimensional structure is sought, is aligned against one or more template sequences, contained in a database. The three dimensional structures for each of the template sequences are known in whole or in substantial part. After each alignment comparison between the query peptide and a template peptide, the method gives a score. After each comparison has been made in the database, the highest scoring alignment pair reflects the optimally aligned query sequence/template sequence(s). The optimal sequence alignment may be used to generate the most accurate structural determinations regarding the query sequence. Still, a query/template alignment producing a sub-optimal score may be used to generate useful structural information regarding the query sequence.

In the second step, referred to as the modeling step, structural information of the query peptide may be predicted based upon structural information corresponding to the sequence or subsequences aligned in the template sequence. The most common of primary sequence homology methods use sequence homologies to predict the three dimensional structure of a query sequence based on the three dimensional structure of aligned template sequences. Still, other primary sequence homology modeling techniques seek to determine primary sequence homology relationships between one or more query sequences based on the primary sequences of aligned template sequences.

The present invention relates to an improved method of performing the first step, namely, an improved method of determining an optimal alignment between a query sequence and a template sequence.

Current, state-of-the-art primary sequence homology modeling techniques such as MODELLER, A. Sali and T. L. Blundell, J. Mol. Biol. 234, 779-815 (1993) require at least 30-40% sequence identity between a query peptide and a template peptide to generate an accurate three dimensional structure. R. Sánchez and A. \check{S}ali, Proc. Natl. Acad. Sci. USA 95, 13597-13602 (1998). With current state-of-the-art methods, less than 20% of the soluble protein residues coded in the Brewer's Yeast genome can be assigned a confident structural model. Id.

MODELLER employs a dynamic programming approach to determining a preferred alignment between a query sequence and a template sequence is typical of the many dynamic programming approaches in the art of sequence alignment. This sequence alignment is then used by MODELLER to construct a three dimensional structure of the query sequence.

Dynamic programming methodologies have been used for determining sequence homologies since they were first introduced by Needleman and Wunsch. S. B. Needleman and C. D. Wunsch, J. Mol. Biol 48., 443-453 (1970); T. F. Smith, M. S. Waterman, Adv. Appl. Math., 2, 482-489 (1981); [M. Gribskov, A. D. McLachlan, and D. Eisenberg, Proc. Natl. Acad. Sci. U.S.A., 84, 4355 (1987); M. Gribskov, M. Homyak, J. Edenfield, and D. Eisenberg, CABIOS 4, (1988); M. Gribskov, D. Eisenberg, Techniques in Protein Chemistry (T. E. Hugli, ed.), p. 108. Academic Press, San Diego, Calif., 1989; M. Gribskov, R. Luthy, and D. Eisenberg, Meth. in Enz. 183, 146 (1990)]. In a general sense, the dynamic programming approaches to determine sequence alignment comprise: (1) creating a matrix composed of the similarity scores for when each pair of residues in the two sequences are matched (a sum matrix), and (2) determining the optimal alignment between the two sequences via constructing a sum matrix using dynamic programming. Numerous variations to detect protein sequence similarity based on the Needleman-Wunsch dynamic programming paradigm have been developed.

In the original Needleman-Wunsch work, only the residue identities between the two proteins were considered in the creation of the sum matrix. More contemporary methods employ a residue substitution scoring system such as point-accepted mutation (PAM) matrices, “A Model of Evolutionary Change in Proteins” in M. O. Dayhoff Ed. Atlas of Protein Sequence and Structure Vol. 5, Suppl. 3, pp. 345-352, 1979, or BLOSUM matrices, S. Henikoff and J. G. Henikoff, Proc. Natl. Acad. Sci. USA 89, 10915-10919 (1992), to generate an alignment sum matrix. Additional information that may used to create an alignment score matrix, include the information from multiple sequence alignments, residue environment profiles (so-called profile threading techniques), secondary structure predictions, and solvent accessibility predictions, to name just a few. S. F. Altschul, T. L. Madden, A. A. Schaffer et al., Nuci. Acids Res. 25, 3389-3402 (1997); J. U. Bowie, R. Lüthy and D. Eisenberg, Science 253, 164-170 (1991); B. Rost, R. Schneider and C. Sander, J. Mol. Biol. 270, 471-480 (1997).

While they employed a very simple sum matrix, the fundamental contribution made by the Needleman-Wunsch work was the application of dynamic programming to determine the optimal global alignment between the two proteins for a given scoring and gap hiearchies (gaps are indicated by residues that are not aligned to another residue in the final alignment, and here “global” means matching the entirety of one sequence and all possible prefixes against substrings of the other). More contemporary approaches have been developed, but they typically involve finding the optimal global, local or global-local alignment path through a sum matrix calculated from the similarity scores in conjunction with gap scores for residues that are not aligned to another residue. D. Fischer and D. Eisenberg, Protein Sci. 5, 947-955 (1996). T. F. Smith and M. S. Waterman, “Identification of Common Molecular Subsequences,” J. Molecular Biology, 147, pp. 195-197, 1981, solved the local alignment problem by introducing a “zero trick”: if an entry of the dynamic programming table is negative, then the optimal local alignment cannot go through this entry because the first part would lower the score; one may therefore replace it with zero, in effect cutting off the prefixes. (This simple trick is known in the computer science art as the maximum subvector method.) O. Gotoh, in “An Improved Algorithm for Matching Biological Sequences,” J. Molecular Biology, 162, pp. 705-708, 1982, then showed that affine gap penalty (separate costs for number and lengths of gaps) is about as efficiently solved as is a linear gap penalty. The identification of multiple, similar segments was achieved by M. S. Waterman and M. Eggert in “A New Algorithm for Best Subsequence Alignments With Application to tRNA-rRNA Comparison,” J. Molecular Biology, 197, pp. 723-728, 1987).

While MODELLER uses a standard dynamic programming procedure to perform an alignment, MODELLER employs various enhancements to improve the final alignment. First, consensus alignments are determined by performing dynamic programming many times using different gap penalties. Second, gap penalties are altered based on the environment of the particular gap, for example, whether or not the gap is located within a template secondary structure (high penalization) or loop region (mild penalization). Even with these additional techniques, MODELLER typically requires at least 30% homology to obtain an alignment of sufficient quality to produce an accurate structural model for a query protein sequence. Another limitation of such homology modeling approaches is that for long loop regions not present in template structures, it is often necessary to use unreliable ab initio or database search methods for modeling such loop regions. Because of these limitations in current homology modeling techniques, there exists a need for improved protein structure prediction methods.

In addition to primary sequence homology modeling programs for predicting three dimensional protein structures such as MODELLER, primary sequence homology modeling programs such as PSI BLAST and HMM also employ sequence alignment methods and consequently have the same limitations as primary sequence homology modeling programs used for predicting three dimensional structures. S. F. Altschul, T. L. Madden, A. A. Schaffer et al., Nucl Acids Res. 25, 3389-3402 (1997); K. Karplus, C. Barrett and R. Hughey, Bioinformatics 14, 846-856 (1998). The current alignment approaches in PSI BLAST and HMM can reliably determine family homologies are structural relationships between a query sequence and a template sequence if there is at least a 30% sequence homology. This is insufficient for many family homology determinations. Divergent evolution causes many proteins in the same structural family to have less than 30% sequence identity, S. A. Teichmann, C. Chothia, and M. Gerstein, Curr. Opin. Struct. Biol. 9, 390-399 (1999), and there are many proteins with sequence identities well below 20% that have very similar structures. It is estimated that nearly two-thirds of the proteins in the protein databank that are believed to not have any structural homologues do in fact have structural homologues. S. E. Brenner, C. Chothia, and T. Hubbard, Curr. Opin. Struct. Biol 7, 369-376 (1997). If these structural homologies and family relationships are to be determined, a sequence alignment method that is accurate at lower levels of sequence homologies is required.

Accordingly, one object of this invention is an improved method of primary sequence homology modeling that is effective with less than 30% sequence homologies. Unlike sequence comparison methods that do not incorporate any structural information in their similarity determinations, the methods according to this invention utilize information from multiple reference sequence alignments with experimentally determined structures to dramatically increase the alignment accuracy between a test sequence and comparison sequence. This increased alignment accuracy greatly enhances the detection of distantly related structural homologues over the state of the art sequence comparison methods and permits accurate structural models to be created for sequences with far less than 30% sequence identity to a sequence of known structure.

As in other alignment methods, the methods for determining a preferred alignment according to the present invention, compare the protein sequence of interest (the query sequence) to a database of comparison sequences or template sequences of known structure in an attempt to recognize a sequence similarity and subsequently construct the structure of the query sequence. However, unlike all previous alignment methods, in the methods according to the invention, a database of reference sequences is pre-analyzed to determine the location of alignment gaps, referred to throughout as bridges and bulges, within each of the templates. In the preferred embodiment, the bridge and bulge information is extracted from multiple sequence alignments between all or substantially all of the reference sequences in a protein structure database (e.g., the Protein Data Bank (PDB)). The database of reference sequences used to determine the bridges/bulges may contain the same sequences as the database of template sequences used for determining a preferred sequence alignment. Methods for determining a pair-wise structure alignment between two protein structures are known to one of skill in the art and include, for example, the Dali method developed by Holm and Sander. Holm, L. and Sander, C. J. Mol. Biol. 233: 123-138 (1993); Holm, L. and Sander, C., Science, 273, 595-602 (1996). The methods according to the invention use the bridge and bulge information to determine an alignment score between the potential alignment sequences of a query sequence and a template sequence. These alignment scores may then be computed between a query sequence and a plurality of template sequences to determine an optimal alignment between a query sequence and a plurality of template sequences.

The alignments generated by methods according to the invention may be used in combination with well-known techniques for assembling a three-dimensional structure from a sequence alignment. One preferred embodiment uses the alignment methods according to the invention to generate a preferred sequence alignment and then uses the comparative modeling package MODELLER, A. Sali and T. L. Blundell, 234 J. Mol. Biol., 779-815 (1993) to generate a predicted three dimensional structure for a query sequence based on this preferred sequence alignment. MODELLER can be understood as combining two methods: 1) first MODELLER determines a preferred sequence alignment of a query sequence to one or more template sequences in a database of template sequences with known three dimensional structures; and 2) next, MODELLER constructs a three dimensional structure of the query sequence based on the input from step 1. Accordingly, the preferred methods of the invention may be used in lieu of MODELLER's sequence alignment methods and in combination with its methods for three dimensional structure construction for an improved combination method for predicting three dimensional structure of a query sequence based homology modeling.

### BRIEF DESCRIPTION OF THE TABLES AND FIGURES

FIG. 1 shows the seven homology sequences found to the query sequence: LVAFADFG-SVTFTNAEATSGGSTVGPSDATVMDIEQDGSVLTETSVSGDS-VTV by the program clustal W.

FIG. 2 represents a similarity matrix which may be formed from the sequence alignment of the two text strings “BIGTOWNSOWN” and “BIGBROWNTOWNOWN.”

FIG. 3 represents a partially completed sum matrix formed from the similarity matrix in FIG. 2 according to the current state-of-the-art sequence alignment methods.

FIG. 4 represents the sum matrix of FIG. 3 at a further stage of completion.

FIG. 5 shows the amount of the GAP penalties that contributed to the gray cells of FIG. 4.

FIG. 6 represents a completed sum matrix for the sequence alignment of the two text strings “BIGTOWNSOWN” and “BIGBROWNTOWNOWN” according to the state-of-the-art current sequence alignment methods.

FIG. 7 represents the highest scoring alignment from FIG. 6 in the PIR format.

FIG. 8 represents schematically the required input data for the methods according to the invention.

FIG. 9 represents a hypothetical BRIDGE/BULGE set for the text strings “BIGTOWNSOWN” and “BIGBROWNTOWNOWN.”

FIG. 10 represents the allowed alignment gaps for the text strings “BIGTOWNSOWN” and “BIGBROWNTOWNOWN” based on the BRIDGE/BULGE set in FIG. 9.

FIG. 11 represents a partially completed sum matrix formed from the similarity matrix in FIG. 2 according to the methods of the current invention.

FIG. 12 represents the sum matrix of FIG. 11 at a later stage of completion.

FIG. 13 shows the amount the gap penalties contributed to the gray cells of FIG. 12.

FIG. 14 represents a completed sum matrix for the sequence alignment of the two text strings “BIGTOWNSOWN” and “BIGBROWNTOWNOWN” according to the methods of the invention.

FIG. 15 represents the highest scoring alignment from FIG. 14 in the PIR format.

FIG. 16 represents the ribbon structure for MG001 as generated by the methods according to the invention.

FIG. 17 represents the optimal sequence alignment between 8C001 and 1b4kA in PIR format as determined by the methods according to the invention.

FIG. 18 shows the crystal structure of law5 on the left and the structure of SC001 on the right as predicted by the methods according to the invention.

FIG. 19 shows a space filling representation of chain A from 1dkf co-crystalized with oleic acid.

FIG. 20 shows the PIR alignment of 1dkf (denoted as gi7766906) and the sequence of chain A of structure 1a28 according to the methods of the invention.

FIG. 21 shows a rainbow ribbon overlay between the predicted structure and the crystal structure of chain A of 1dkf.

FIG. 22 shows an overlay of the predicted structure according to the methods of the invention 1dkf and the crystal structure for 22 key residues that form the oleic acid binding pocket.

FIG. 23 shows a stick diagram of 1a252 (PDB code) co-crystallized with estradiol. The estradiol ligands are shown in space filling format.

FIG. 24 shows the alignment according to the methods of the invention in PIR format between the sequence of the estrogen receptor (denoted as gi365993 1) and the sequence of chain A of structure 1a28, denoted 1a28A.

FIG. 25 shows a rainbow ribbon overlay between the predicted structure according to the methods of the invention of the estrogen receptor and the crystal structure of chain A of 1a52.

FIG. 26 shows an overlay of the predicted structure according to the methods of the invention for estrogen receptor and the crystal structure for 19 key residues that form the estradiol binding pocket.

FIG. 27 shows the alignment formed from the methods of the invention in PIR format between the sequence of halorhodopsin, denoted 1e12A, and the sequence of bacteriorhodopsin, denoted 1c3wA made by the methods according to the invention.

FIG. 28 shows a rainbow ribbon overlay between the three-dimensional structure created using the alignment in FIG. 27, compared to the halorhodopsin crystal structure, chain A of PDB code 1e12.

FIG. 29 shows the alignment, formed from the methods according to the invention, in PIR format, between the sequence of bacteriorhodopsin, denoted 1c3wA, and the sequence of rhodposin, chain A of PDB structure 1f88, denoted lf88A.

FIG. 30 shows a rainbow ribbon overlay between the three-dimensional structure created using the alignment in FIG. 29, compared to the bacteriorhodopsin crystal structure, chain A of PDB code 1 c3w.

FIG. 31 shows the alignment, formed from the methods according to the invention, in PIR format, between the sequence of a membrane spanning chain of the photosynthetic reaction center, denoted 6prcM, and the sequence of a different chain from the photosynthetic reaction center, chain L of PDB structure 6prc, denoted 6prcL.

FIG. 32 shows a rainbow ribbon overlay between the three-dimensional structure created using the alignment in FIG. 31, compared to the crystal structure for chain M of PDB code 6prc.

FIG. 33 shows the alignment according to the invention in PIR format between the sequence of ompA, denoted 1bxwA, and the sequence of ompX, chain A of PDB structure 1qj8, denoted 1qj8A.

FIG. 34 shows a rainbow ribbon overlay between the three-dimensional structure created using the alignment in FIG. 33, compared to the ompA crystal structure, chain A of PDB code 1bxw.

FIG. 35 shows the alignment according to the invention in PIR format between the sequence of ompK36, denoted 1osmA, and the sequence of porin protein 2por.

FIG. 36 shows a rainbow ribbon overlay between the three-dimensional structure created using the alignment in FIG. 35, compared to the ompK36 crystal structure, chain A of PDB code 1 osm.

FIG. 37 shows the alignment, formed from the methods according to the invention, in PIR format, between the sequence of sucrose-specific porin, denoted 1a0tP, and the sequence of maltoporin, chain A of PDB structure 2mpr, denoted 2mprA.

FIG. 38 shows a rainbow ribbon overlay between the three-dimensional structure created using the alignment in FIG. 37, compared to the sucrose-specific porin crystal structure, chain P of PDB code 1a0tP.

Table 1 lists the structure alignment between domains 1ovaA and 1by7A.

Table 2 provides a BRIDGE/BULGE gap list of bridges and bulges for the domain 1ovaA derived from DALI structure alignments between 1ovaA and the protein domains 1ova, 1ovaC, 1azxI, and 1by7A.

Table 3 provides a comparison of the advantages of the methods of the present invention versus the state-of-the-art methods.

Table 4 shows the relative abilities of the alignment methods of the present invention and PSI Blast to recognize sequence homology relationships at the Family, Superfamily, Fold and Class levels for 27 sequences in the SCOP database.

Table 5 shows the number of residues correctly modeled using the alignment methods according to the invention for 34 previously unmodeled Mycoplasma genitalium sequences.

Table 6 provides a comparison between predicted structures using the alignment methods according to the invention with the ModBase database for the first 180 sequences in the Mycoplasma genitalium genome. The number of residues built into a reliable structural model is given in each column. Substantially complete models containing at least 80% of the total sequence length are highlighted in bold. Structures generated by each method passed identical reliability tests. These tests are published (Sanchez and Sali 1998), and represent a threshold where the structures will have the correct fold with a confidence limit of >95%.

Table 7 provides PDB structures found to have sequence similarity to SC001 by gapped-BLAST.

Table 8 provides a partial list of bridges and bulges for the domain 1ovaA derived from DALI structure alignments between 1ovaA and the listed protein domains.

### SUMMARY OF THE INVENTION

A preferred embodiment of the invention is a method for determining a preferred sequence alignment between a query sequence and at least one template sequence comprising the steps of: 1) aligning two or more reference sequences to determine one or more BRIDGE/BULGE gaps; 2) determining an alignment score between each potential alignment of the query sequence and each template sequence based on whether or not a given sequence alignment between the query sequence and each template sequence creates a BRDIGE/BULGE gap and 3) determining a preferred sequence alignment based on the alignment scores of the query sequence with each template sequence. A preferred sequence alignment includes any sequence alignment that may be used to determine useful structural information regarding the query sequence. The optimal sequence alignment is the alignment with the highest score. Although, an optimal sequence alignment may be used to generate the most accurate structural information regarding the query sequence, often sequence alignments with sub-optimal sequences still provide useful structural information and primary sequence homology relationships.

Another embodiment of the invention is a method for determining a preferred alignment between a query sequence and a template sequence comprising the steps of: 1) aligning two or more reference sequences to determine one or more reference alignment gaps known as BRIDGE/BULGE gaps; 2) forming a sequence alignment similarity matrix for the query sequence and one or more template sequences; 3) determining a sequence alignment sum matrix from the dynamic evolution of each sequence alignment similarity matrix based on whether the alignment of the query sequence with each template sequence creates a BRIDGE/BULGE gap; and 4) determining a preferred alignment between the query sequence and each template sequence from the dynamic evolution of each sum matrix.

Another embodiment of the invention is method for determining the three dimensional structure of a query sequence based upon primary sequence homology modeling with one or more template sequences using the methods of the invention for determining an optimal sequence alignment. When the preferred alignment methods according to the invention are used in combination with primary sequence homology modeling methods to predict the three dimensional structure of a query sequence or determine the primary sequence homology relationships of a plurality of query sequences, it is possible to generate accurate structural models of query sequences at lower alignment homologies than the current state-of-the-art permits. Accordingly, another embodiment of the invention is a method for predicting three dimensional structure of query sequences using primary sequence homology modeling methods when the query sequence and template contain from 10-20% homologous residues. A still further embodiment of the invention is a method for determining the primary sequence homology relationships for at least two query sequences using primary sequence homology modeling methods when the query sequence and template from 10-20% homologous residues.

### DETAILED DESCRIPTION OF THE INVENTION

A preferred embodiment of the invention is a method for determining a preferred sequence alignment between a query sequence and one or more template sequences comprising the steps of: 1) aligning two or more reference sequences to determine one or more reference alignment gaps known as BRIDGE/BULGE gaps; 2) determining an alignment score between each potential alignment of the query sequence and each template sequence based on whether or not a given sequence alignment between the query sequence and each template sequence creates a BRIDGE/BULGE gap and 3) determining a preferred sequence alignment based on the alignment scores of the query sequence with each template sequences.

Preferred Methods for Determining Reference Alignment Gaps-BRIDGE/BULGE Gaps

In a preferred method of the invention, a list of reference alignment gaps known as a BRIDGE/BULGE list, is generated from aligning each reference sequence in a database of reference sequences against every other reference sequence. Preferably, such a database of reference sequences includes all or a statistically significant cross section of the know protein sequences such as the continuously evolving Protein Data Bank (PDB). Such structure comparison techniques are known to one of skill in the art and include, for example, the Dali method developed by Holm and Sander, the Combinatorial Extension Method (CE), and VAST. Holm, L. and Sander, C. J. Mol. Biol. 233, 123-138 (1993); Holm, L. and Sander, C., Science 273, 595-602 (1996); Shindyalov, I. N., and Bourne, P. E., Protein Eng. 11, 739-747 (1998); Gibrat, J-F., Madei, T. and Bryant, S. H., Curr. Opin. Struct. Biol. 6, 377-385 (1996).

TABLE 1

| | 1ovaA | 1by7A |
|---|---|---|
| Aligned | 1-63 | 1-63 |
| Gap | (64) | |
| Aligned | 65-68 | 64-67 |
| Gap | (69-68) | |
| Aligned | 79-91 | 68-80 |
| Gap | (92-97) | |
| Aligned | 98-189 | 81-172 |

Table 1 shows a structure alignment produced by the program Dali for the protein domains 1ovaA and 1by7A (the C-terminus of the alignment has been truncated at residue 189 of 1ovaA). As Table 1 suggests when two sequences are aligned, often large regions of the two sequences are identical and are separated by regions where the amino acid residues differ. In particular, when 1ovaA is aligned against 1by7A, the first 63 and the last 91 residues match between the two sequences. The intervening regions alternately align and do not align over short sequence lengths. For example, residues 69-78 in 1ovaA do not align to any residues in 1by7A, even though the structures are similar on both sides of the gap. Thus, with respect to 1by7A, 1ovaA has a 9-residue bulge in this region. Conversely, with respect to 1ovaA, the structure 1by7A bridges 9 residues in this region of 1ovaA.

It is well known in the art that a structure comparison database can be constructed for each protein relative to the entire database. See e.g. FSSP database, Holm and Sander, Science 273, 595-602 (1996). Given a set of sequence alignments, it is possible to generate a list of all of the bridges and bulges that occur in the various sequence alignments with respect to a given structure. In general, results according to the methods of the invention are generally improved as the number of sequences and genomes contained within the database used to determine BRIDGE/BULGE information are increased. Table 2 shows a partial list of the bridge and bulge information that can be derived from aligning various sequences in the Protein Databank (PDB). F. C. Bernstein, T. F. Koetzle, G. J. B. Williams et al. J. Mol. Biol. 112, 535-542 (1977); H. M. Berman, J. Westbrook, Z. Feng, G. Gilliland, T. N. Bhat, H. Weissig, I. N. Shindyalov, P. E. Bourne Nucleic Acids Research, 28: 235-242 (2000); WWW address:htt://www.rcsb.org/pdb] to the protein domain 1ovaA. The bridges that have been derived from the alignment of 1ovaA with 1by7A in Table 1 are highlighted in gray.

TABLE 2

| Template | Gap | Start Res. | End Res. | # Res. In |
|---|---|---|---|---|
| Protein | Type | In 1ovaA | In 1ovaA | Template |
| 1ovaC | BRIDGE | 341 | 354 | 1 |
| 1ovaB | BRIDGE | 65 | 79 | 1 |
| 1azxI | BULGE | 24 | 25 | 2 |
| 1azxI | BULGE | 62 | 63 | 3 |
| 1azxI | BRIDGE | 66 | 78 | 1 |
| 1azxI | BULGE | 92 | 94 | 3 |
| 1azxI | BRIDGE | 223 | 225 | 1 |
| 1azxI | BRIDGE | 269 | 272 | 1 |
| 1azxI | BULGE | 308 | 309 | 2 |
| 1azxI | BULGE | 316 | 317 | 3 |
| 1azxI | BULGE | 338 | 341 | 8 |
| 1azxI | BRIDGE | 345 | 348 | 2 |
| 1azxI | BRIDGE | 351 | 353 | 1 |
| 1by7A | BRIDGE | 63 | 65 | 1 |
| 1by7A | BRIDGE | 68 | 79 | 1 |
| 1by7A | BRIDGE | 91 | 98 | 1 |
| 1by7A | BRIDGE | 189 | 193 | 1 |
| 1by7A | BULGE | 235 | 237 | 1 |
| 1by7A | BULGE | 249 | 250 | 5 |
| 1by7A | BULGE | 308 | 309 | 2 |
| 1by7A | BRIDGE | 339 | 355 | 1 |

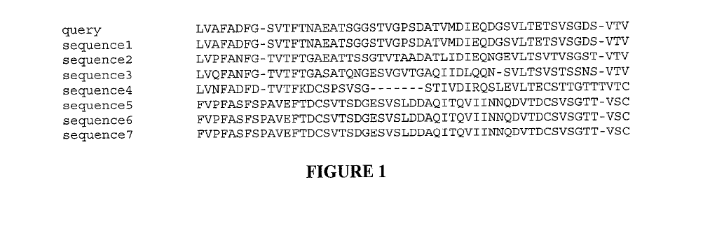

Another preferred method for determining BRIDGE/BULGE information employs an algorithm such as BLAST, S. F. Altschul, W. Gish, W. Miller, E. W. Meyers, and D. J. Lippman, J. Mol. Biol. 215, 403-410 (1990), to determine a set of homology sequences to the query sequence and the template sequences from any large sequence database that contains a statistically representative cross section of many sequences across multiple genomes. Preferably the databases that are used to determine the BRIDGE/BULGE lists according to this preferred embodiment include all the known sequences with homologies of at least 45% to the query and template sequences. A suitable database would be the non-redundant protein sequence databank at the NIH, which currently contains more than 600,000 sequences from more than 100 different organisms. A BRIDGE/BULGE list may then be determined from the sequence homology sets formed from query sequence and the template sequences using any multiple sequence alignment algorithm known in the art, such as clustalW, J. D. Thompson, D. G. Higgins, T. J. Gibson, Nucl. Acids Res. 22, 4673-4680 (1994). FIG. 1 shows the 7 homology sequences found (performed by clustalW) for the sequence:

LVAFADFGSVTFTNAEATSGGSTVGPSDATVMDIEQDGSVLTETSVSGDSVTV.

With respect to the query sequence, the multiple sequence alignment contains 2 different one-residue bulge regions, represented by the “G-S” and “S-V” points in the query sequence. The multiple alignment in FIG. 1 also contains one bridge region, where the residues “STVGP SD” in the query sequence are bridged by a gap region in sequence 4. Note that if three-dimensional models of the homology sequences exist it is possible to verify that each of the bridges and bulges found comply with the physical limitations imposed by the three dimensional structures.

An alternative source of a BRIDGE/BULGE list consists of a list of bridge and bulge gaps that comply with the physical limitations imposed by the 3-dimensional protein structure. For example, a list of inter-residue distances between the C-alpha carbons in each residue in the template sequence can be created. Inter-residue distances that lie between certain thresholds can be considered candidates for an appropriate BRIDGE/BULGE gap. For instance, two-residues that are approximately 5 Å apart are excellent candidates to be separated by one residue. A bridge of one residue at this point in the structure would not disrupt the overall fold, and could be considered for inclusion in the BRIDGE/BULGE gap set (if these residues are indeed separated by more than one residue in the query structure). In this manner, a set of bridges and bulges that do not disrupt the 3-dimensional structure of the template sequence may also be used in a BRIDGE/BULGE gap set.

The structure of intra-membrane proteins, located all or in part in the cell membrane, have a number of unique characteristics that differentiate them from their soluble protein counterparts. One such characteristic is the high degree of structural homology exhibited by membrane proteins for the regions of the protein that lie within the membrane. Conversely, the intra- and extra-cellular loops in these proteins are known to be quite flexible and not nearly as structurally conserved. The methods of the current invention are uniquely suited to model such sequences. Given a membrane protein template structure, the intra- and extra-cellular loop regions can be identified, and the list of BRIDGE/BULGE gaps for the membrane template can be enriched so that all possible loop lengths are present in the candidate alignment set. Furthermore, BRIDGE/BULGE gaps which disrupt the highly conserved intra-membrane structure of the protein can be removed from the BRIDGE/BULGE set, so that only sequence alignments which preserve this highly conserved structure are considered in the optimal alignment. The parameters for standard gap opening and extension, as well as BRIDGE/BULGE gap opening and extension should be determined for membrane proteins independently from soluble proteins.

A list of bridges and bulges contains valuable information regarding the types of gaps that are known to exist in nature for a given sequence comparison. In the preferred methods of the invention, each gap listed in the BRIDGE/BULGE set is given an opportunity to participate in determining the optimal alignment between a query sequence and a template sequence. The current methods in the art for determining an optimal sequence alignment between a query sequence and a template sequence do not consider whether a proposed alignment gap is found elsewhere in nature.

One skilled in the art will quickly appreciate why such consideration is important. When comparing two sequences, as the relative sequence homology falls, the frequency and sizes of alignment gaps typically increases. Without consideration of whether or not there is any physical basis to the gaps, the determination of optimal alignment becomes disconnected from physical reality of the three dimensional structure of the sequence.

Preferred Methods for Calculating a Sequence Alignment—the Sum Matrix

A preferred method for determining an optimal sequence alignment between a query sequence and a template sequence comprises dynamically evolving a sequence similarity matrix to calculate a sum matrix according to an algorithm that considers whether or not a proposed alignment gap creates a known BRIDGE/BULGE gap. Although the use of similarity matrices and dynamic programming are commonly employed in current alignment techniques, current alignment techniques do not determine an optimal alignment by reference to whether or not a proposed BRIDGE/BULGE gap physically exists.

### EXAMPLE 1

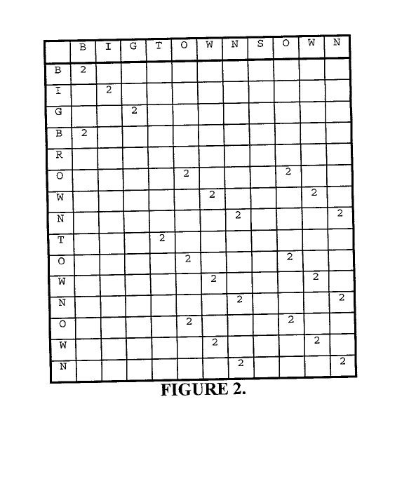

Example 1 shows the current method for determining an optimal sequence alignment by dynamically evolving a similarity matrix to calculate a sum matrix. FIG. 2 shows an exemplary similarity matrix constructed for the two sequences “BIGTOWNSOWN” and “BIGBROWNTOWNOWN”, using a very simple scoring function such that s_{i,j}=2 if the letters at matrix positions i and j are the same and s_{i,j}=0 if the letters at matrix positions i and j are different.

In dynamic programming, the sum matrix may be calculated from dynamically evolving a similarity matrix. An exemplary evolution scheme for connecting the elements of a similarity matrix sᵢⱼ to the elements of a sum matrix Sᵢⱼ is shown in Equation 1.

Sᵢⱼ = sᵢⱼ + Max{

| | |
|---|---|
| S_{i+1, j+1}, | [Diagonal, down and to the right] |
| S_{i+1, j+2 to jmax} − GAP, | [Down row i+1, all possible gaps] |
| S_{i+2 to imax, j+2} − GAP, | [Down column j+1, all possible gaps] |

},

where s_{i,j} denotes the score of cell (i, j) in the similarity matrix, and Max denotes the maximum value for the three terms in the bracketed expression. GAP represents the gap penalty for the proposed gap opening and extension. An exemplary GAP scoring penalty is shown in Equation 2.

GAP=Open−k(extension), (2)

where “Open” represents a penalty constant for opening a gap and “k(extension)” is a constant representing the penalty constant for extending the gap “k” residues.

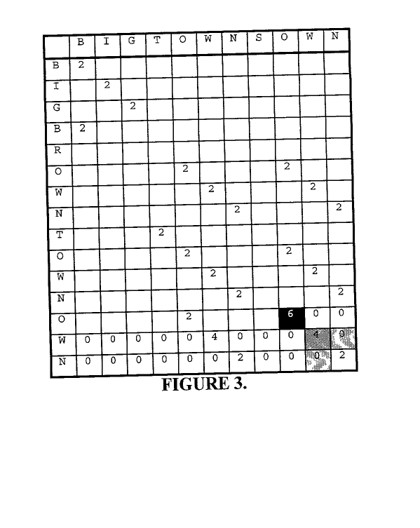

A typical dynamic programming algorithm begins filling in the sum matrix from the bottom row, and continues moving up the matrix, filling in the scores for each cell in the row from right to left. FIG. 3 shows the sum matrix being constructed, where the gap opening and extension penalties are 2 and 1, respectively. The sᵢⱼ=2 scores from the similarity score matrix have already been transferred to the sum matrix in this example. In FIG. 3, the bottom two rows of the sum matrix have been completed, and the third row from the bottom is being complete. The matrix elements that are gray shaded represent the matrix elements that are considered when determining the score of the black matrix element. The darkest of the gray scaled matrix elements along the diagonal is the matrix element that contributes to the value of the black matrix element.

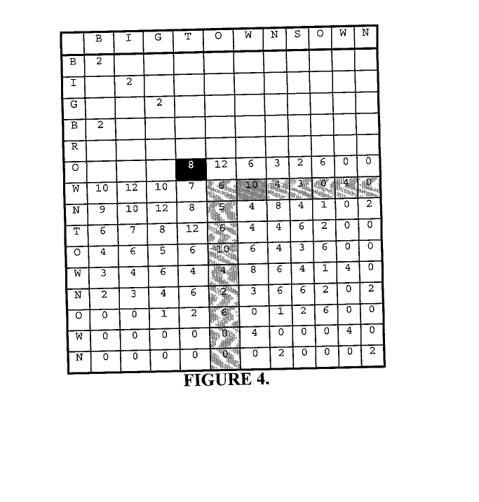

FIG. 4 shows the sum matrix at an even further stage of development, this time with the nine bottom rows completed. As above, the gray shaded matrix elements are the positions considered when determining the score in the black shaded matrix element. In this case, the highest score comes from the darkest gray shaded element that is two columns away from the black cell.

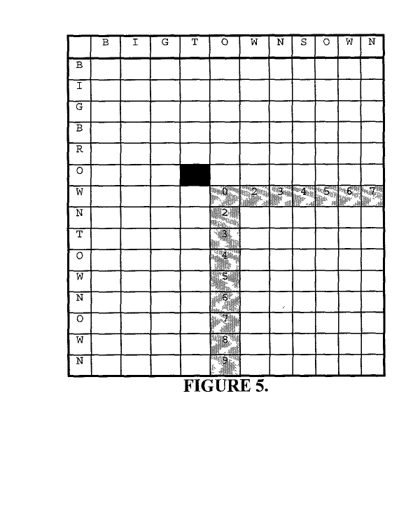

FIG. 5, shows the GAP penalties that are used in equation (1) for the gray cells that are alignment candidates for the black-shaded cell from FIG. 4. The cell directly below and to the right of the black-shaded cell has GAP=0. There are two cells with GAP=2, where the gap is first opened but not extended. Cells further from the black-shaded cell then also receive an extension penalty of 1, and so their overall GAP penalty increases by one unit as the length of the extension increases k from equation 1).

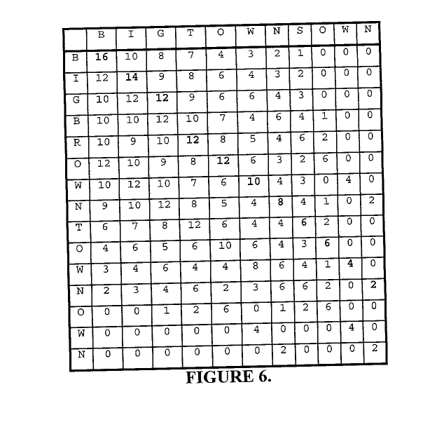

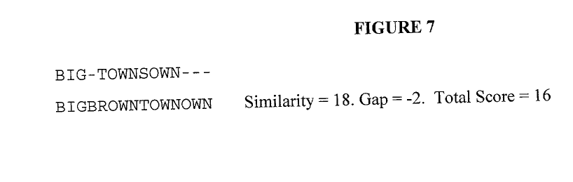

FIG. 6 shows the completed sum matrix formed from the dynamic evolution of the similarity matrix with matrix elements sᵢⱼ as defined above. Once the sum matrix is completed, the optimal alignment is found by finding the highest scoring cell among all cells in the top row and left most column of the sum matrix, and then tracing back through the cells that led to this maximum scoring cell. In this example, the top left optimal alignment begins in the top left cell and is highlighted in bold. The highest scoring alignment is shown in FIG. 7 outside the context of the sum matrix in the widely used PIR format.

The current dynamic programming methods as taught above and as typified by Equation 2, do not consider BRIDGE/BULGE information when evolving a similarity matrix to calculate the sum matrix. Thus, the current methods for determining an optimal sequence alignment between a query sequence and template sequence make such a determination without reference to whether a proposed BRIDGE/BULGE has a physical basis in nature. This has important implications when making sequence comparisons between two sequences with low sequence homologies and explains why the current alignment techniques fail at low homologies. When comparing two sequences, as the relative sequence homology decreases, the relative gap sizes and frequency increase. Without consideration of whether or not the gaps have any precedent in nature, the determination of optimal alignment becomes disconnected from physical reality.

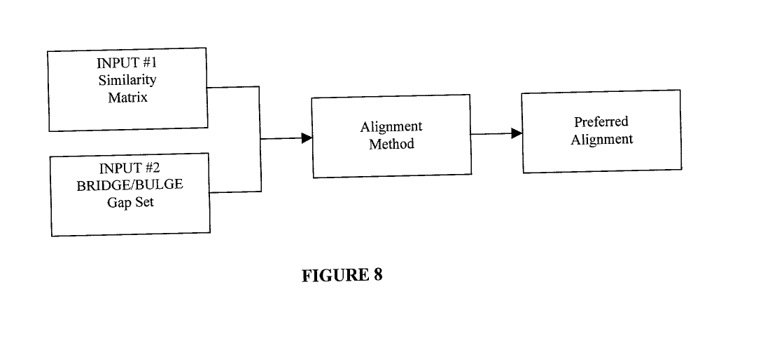

The methods of the present invention are based on the realization that if the dynamic programming scheme of a similarity matrix to form a sum matrix is going to be accurate at low sequence homologies, the dynamic programming scheme must consider whether or not a proposed alignment has precedence in nature. The preferred methods of the invention, like the current methods for determining an optimal sequence alignment between a query sequence and a template sequence, use dynamic programming to output a sum matrix from an input similarity matrix. However, the present methods for determining an optimal sequence alignment also consider one more input variable, namely, whether or not any BRIDGES/BULGES in a proposed alignment have any physical basis in nature. FIG. 8 pictorially shows the two basic inputs required for the methods according to the invention.

In a preferred method according to the invention, a similarity matrix with matrix elements sᵢⱼ is dynamically evolved according to Equation 3 to calculate the sum matrix with matrix elements Sᵢⱼ.

Sᵢⱼ = sᵢⱼ + Max{

| | |
|---|---|
| S_{i+1, j+1}, | [Diagonal, down and to the right] |
| S_{i+1, j+2 to jmax} − GAP, | [Down row i+1, all possible j] |
| S_{i+2 to imax, j+2} − GAP, | [Down column j+1, all possible i] |

| | |
|---|---|
| S_{m,n} − BRIDGE/BULGE | [Bridges and bulges that terminate sum |

matrix element i,j]

},

The terms in Equation 3, are defined the same as the terms in Equation 2 with the additional term BRIDGE/BULGE. BRIDGE/BULGE corresponds to the penalty for a known bridge or bulge that begins at the m, n matrix element of the sum matrix and ends at the i, j matrix element of the sum matrix. Max{S_{i+1, j+1}, S_{i+1, j+2 to jmax}−GAP, S_{i+2 to imax, j+2} −GAP, S_{m, n}−BRIDGE/BULGE} refers to the maximum value of the four terms contained within the brackets. The similarity matrix may be developed by any of the methods known in the art.

### EXAMPLE 2

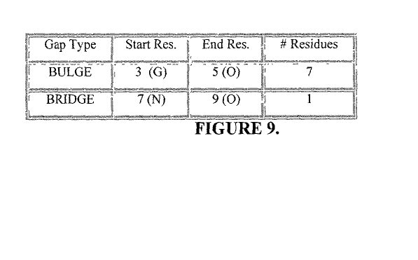

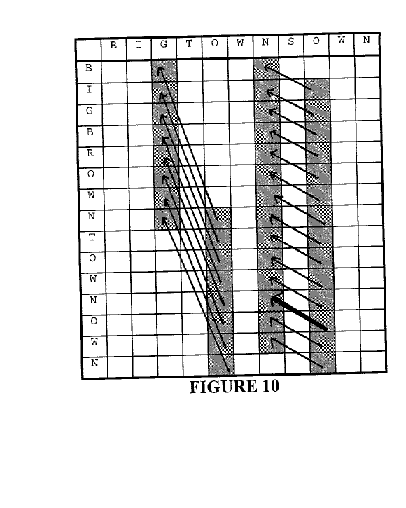

Example 2 demonstrates how the inclusion of BRIDGE/BULGE information from the preferred method described by Equation 3 affects the determination of a preferred alignment between “BIGTOWNSOWN” with “BIGBROWNTOWNOWN” based on the similarity matrix in FIG. 2 and the BRIDGE/BULGE set in FIG. 9. For the purposes of this calculation, gap opening and extension penalties for gaps that are not present in the known BRIDGE/BULGE set are 3 and 2, respectively, and the gap opening and extension penalties for gaps that are present in the known BRIDGE/BULGE set are 1 and 0, respectively. FIG. 10 shows the bridge and bulge gaps that are allowed by the BRIDGE/BULGE gap set in FIG. 9. Thus, FIG. 10, shows how a BRIDGE/BULGE set controls the dynamic evolution of the sum matrix from a similarity matrix.

The preferred methods of the invention initially proceed by filling in the sum matrix beginning with the bottom row, and moving up the matrix, filling in the scores for each cell in the row from right to left.

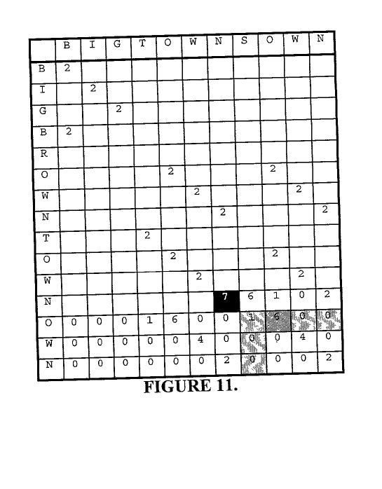

In FIG. 11, the bottom three rows of the sum matrix have been completed, and the fourth row from the bottom is being filled in. Once again, the gray shaded matrix elements are the potential matrix elements considered when determining the score in the black shaded matrix elements and the darkest gray shaded matrix element is the matrix element that actually contributes to the score of the black matrix element. As is shown in FIG. 10 by the thickest arrow, the transition from the dark gray matrix element to the black is permitted by the BRIDGE/BULGE set shown in FIG. 9.

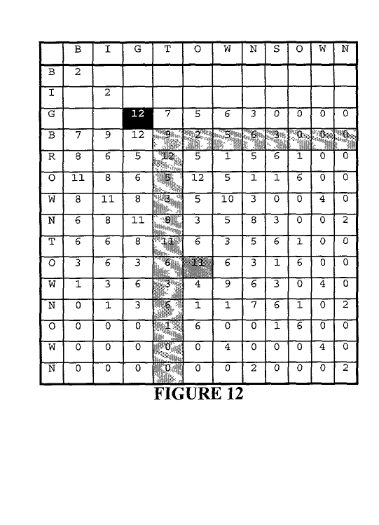

FIG. 12 shows the sum matrix at an even further stage of development with the bottom twelve rows completed. As above, the gray shaded matrix cells are the positions considered when determining the score in the black shaded cell. In this case, the highest score comes from the dark gray shaded cell that is in the BRIDGE/BULGE gap set.

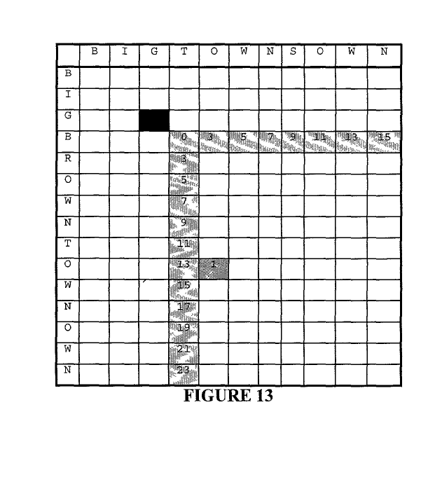

FIG. 13, shows the GAP penalties that are used in Equation 2 for the gray cells that are alignment candidates for the black-shaded cell from FIG. 12. The transition from the darker gray cell to the black cell is in the BRIDGE/BULGE gap set and is thus has a gap penalty of 1.

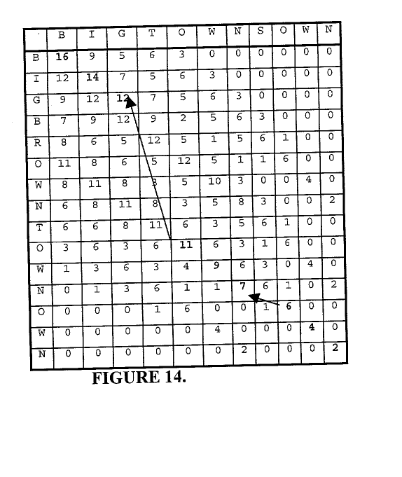

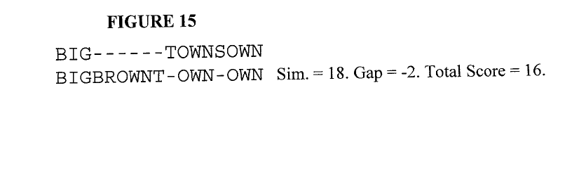

FIG. 14 shows a sum matrix according to a preferred method of the invention for the hypothetical alignment of “BIGTOWNSOWN” with “BIGBROWNTOWNOWN”. Once the sum matrix is completed, the optimal alignment may be found by finding the highest scoring cell among all cells in the top row and left most column of the sum matrix, and then tracing back through the cells that led to this maximum scoring cell. For this example, the optimal alignment begins in the top left cell and is highlighted in bold. Arrows have been used to designate the gaps in the optimal alignment that are listed in the BRIDGE/BULGE gap set. Note that the globally optimal alignment obtained in this case is different from the standard dynamic programming alignment obtained in FIG. 6. The highest scoring alignment is shown in FIG. 15 outside the context of the sum matrix in the widely used PIR format. From FIG. 15, it is evident that the highest scoring alignment obtained in this example does not continuously align the residues from either the query sequence or the template sequence, since the bulge gap present in the final alignment leaves out residues in both sequences.

Preferred Methods for Determining BRIDGE/BULGE Penalties

Methods for determining the gap opening and extension penalties in dynamic programming are well known in the art. A preferred method is to empirically tune these parameters to produce the optimal results for a large number of protein sequences where the optimal alignment is known. A common procedure is to compile the results for many different gap opening and extension penalty combinations then choose the parameters that perform the best over the test set. This procedure is taught for example, in B. Rost, R. Schneider and C. Sander, J. Mol. Biol. 270, 471-480 (1997). When paramaterizing a standard dynamic programming procedure for optimizing sequence alignment, the two variables that must be parametized are the gap opening and gap extension penalties. In the methods according to the invention, in addition to the standard gap opening and gap penalty parameters, penalties for the BRIDGE/BULGE set gap opening and extension penalties must also be parameterized. These parameters can be tuned using the same methods used to determine the standard gap opening and extension penalties used for dynamic programming.

Preferred Combination Methods for Determining Three Dimensional Structures and Family Homologies

Once an alignment is constructed between a query sequence and a protein structure template or templates, there are a variety of sequence homology modeling methods well known in the art for constructing the 3-dimensional structures of the query sequence. One widely used method is rigid-body assembly wherein the precise coordinates of the backbone residues of the template proteins are used as coordinates for the corresponding aligned residues in the query protein. K. Brew, T. C. Vanaman, and R. C. Hill, J. Mol. Biol. 42, 65-86 (1969); T. L. Blundell, B. L. Sibanda, M. J. E. Sternberg, and J. M. Thornton, Nature 326, 347-352 (1987); W. J. Browne, A. C. T. North, D. C. Phillips, J. Greer, Proteins 7, 317-334 (1990). Another set of methods familiar to the art is segment-matching methods, which rely on the approximate coordinates of the atoms in the template proteins. T. H. Jones, S. Thirup, EMBO J. 5, 819-822 (1986); M. Claessens, E. V. Cutsem, I. Lasters, S. Wodak, Protein Eng. 4, 335-345 (1989); R. Unger, D. Harel, S. Wherland, J. L. Sussman, Proteins 5, 355-373 (1989); M. Levitt, J. Mol. Biol. 226, 507-533 (1992)]. Yet another group of methods does not explicitly use the coordinates of the template proteins, but uses the templates to generate a set of inter-residue distance restraints used to create the query structure. Given the set of restraints, methods such as distance geometry or energy optimization techniques are used to generate a structure for the query that satisfies all of the restraints. T. F. Havel and M. E. Snow, J. Mol. Biol. 217, 1-7 (1991); S. M. Brockelhurst, R. N. Perham, Prot. Science 2, 626-639 (1993); A. Sali and T. Blundell, J. Mol. Biol. 234, 779-815 (1993); S. Srinivasan, C. J. March, and S. Sudarsaman, Protein Eng. 6, 501-512 (1993); A. Aszodi and W. R. Taylor, Folding Design 1, 325-34 (1996)]. It is widely known in the art that the accuracy and precision of each of the three classes of algorithms is similar for a given query-template alignment.

The methods of the present invention may also be used to determine relative homology relationships between a plurality of query sequences. A preferred method for determining the relative homology relationships between a plurality of query sequences comprises determining an optimal alignment score of each query sequence against one or more template sequence and determining a relative homology between the query sequences by comparing the preferred alignment scores. Query sequences with alignment scores to one or more of the same template sequences may be considered more closely related than query sequences with more divergent alignment scores.

Advantages to the Preferred Methods of the Invention Relative to Current Methodologies

In the preferred methods, an optimal sequence alignment between a query sequence and a template sequence is determined by reference to whether a proposed bridge or bulge has precedence in nature. Because every bridge and bulge gap used in constructing the alignment exists within the three-dimensional database, it is known that all of the gaps can be satisfied by a three-dimensional protein model void of molecular geometry violations (i.e., the gaps are physical).

Furthermore, because the preferred methods use the bridge and bulge information from known structures, appropriate conformations for long bridge and bulge gaps already exist among the sequences in the PDB. This represents an enormous benefit over current state-of-the art methods. For example, in the alignments produced by the MODELLER program, the only way all of the residues in a query sequence will have a structural template is if enough structural templates are included so that all of the different loop length variations are considered. With the methods of the present invention, the structural templates required to achieve such a task are pre-determined, before the final consensus alignment process begins. This leads to much more accurate predictions in gapped regions, since loop building by ab initio or database search methods is rarely required (such methods commonly lead to poorly modeled or miss-oriented structural regions). These enhancements are summarized in Table 3.

TABLE 3

| | State-of-the-art | STRUCTFAST |
|---|---|---|
| Alignment Step | No-guarantee gaps are | Bridge/Bulge gaps known |
| | physical | to be physical |
| Gap Building | Ab initio or database search | Structural templates for |
| Step | loop construction | Bridge/Bulge gaps already |
| | | known. |

In the following examples, the methods of the current invention will be compared against the state-of-the-art alignment techniques to solve various structural homology modeling problems.

### EXAMPLE 3

Example 3 tests the methods of the invention relative to the PSI-BLAST algorithm, S. F. Altschul, T. L. Madden, A. A. Schaffer et al., 25 Nucl. Acids Res., 3389-3402 (1997), to detect sequentially distant structural homologues. PSI-BLAST currently represents the state-of-art in homology modeling programs. E. Lindahl and A. Elofsson, 295 J. Mol. Biol., 613-625 (2000). Using a test procedure outlined by Lindahl and Elofsson and a set of 27 known protein sequences, in this Example, each algorithm was tested to determine its relative ability to recognize structural neighbors with less than 25% sequence homology at the family, superfamily, fold, and class levels of structural similarity (family being the closest relationship, fold being the weakest) as defined in the SCOP protein database, A. G. Murzin, S. E. Brenner, T. Hubbard and C. Chothia, J. Mol. Biol., 247, 536-540 (1995). All of the structural similarities in the test set also exist in the FSSP database, Holm and Sander, 273 Science, 595-602 (1996), so that regions of high structural homology were ensured to exist even at the fold and class level of similarity. Overall, there were 99 family, 171 superfamily, 184 fold, and 1931 class relationships in the test. The ability of the preferred methods and PSI-BLAST to recognize these relationships with an overall rank of 1, 5, and 10 (i.e. 0, 4, and 9 false positives) are shown in Table 4. These results demonstrate a dramatic increase in sequence recognition capabilities at the superfamily, fold and class similarity levels using the methods according to the invention.

TABLE 4

| | STRUCTFAST/PSIBLAST | | |
|---|---|---|---|
| | Rank 1 | Rank 5 | Rank 10 |
| FAMILY | 54/51% | 61/55% | 62/59% |
| SUPERFAMILY | 18/12% | 33/17% | 37/20% |
| FOLD | 3/0% | 10/1% | 37/1% |
| CLASS | 3/1% | 9/1% | 13/2% |

### EXAMPLE 4

Example 4 demonstrates that the methods of the invention, in combination with widely available homology modeling packages, may be used to predict the three dimensional structure of a query sequence. In this example 54 query sequences from the Mycoplasma genitalium genome cannot be assigned an accurate structural model using the state-of-the-art alignment techniques in MODELLER, A. \check{S}ali and T. L. Blundell, J. Mol. Biol., 234, 779-815 (1993) alone, were modeled using the alignment methods of the invention in combination with three dimensional structure generating portion of MODELLER. The results of this experiment are summarized in Table 5. Table 5 shows that when the methods of the invention are used to generate preferred sequence alignments and MODELLER is used to generate the three dimensional protein structures based on these preferred alignments, 35 out of the 54 sequences (65%), representing 8,800 previously unmodeled residues, were successfully modeled as judged by the pG test, R. Sánchez and A. \check{S}ali, “Large-scale protein structure modeling of the Saccharomyces cerevisiae genome”, Proc. Natl. Acad. Sci. USA, 95, 13597-13602 (1998)], employing Z-scores from PROSAII, M. J. Sippl, Proteins, 17, 355-362 (1993).

TABLE 5

| GENOME SEQUENCE | # OF RESIDUES MODELED |
|---|---|
| MG006 | 210 |
| MG013 | 292 |
| MG021 | 501 |
| MG036 | 491 |
| MG042 | 131 |
| MG063 | 244 |
| MG065 | 236 |
| MG080 | 125 |
| MG083 | 185 |
| MG090 | 93 |
| MG094 | 264 |
| MG106 | 186 |
| MG108 | 260 |
| MG112 | 209 |
| MG154 | 140 |
| MG155 | 72 |
| MG166 | 166 |
| MG180 | 241 |
| MG187 | 139 |
| MG235 | 281 |
| MG253 | 265 |
| MG254 | 308 |
| MG268 | 210 |
| MG273 | 322 |
| MG274 | 329 |
| MG280 | 165 |
| MG303 | 238 |
| MG327 | 238 |
| MG329 | 257 |
| MG377 | 149 |
| MG378 | 508 |
| MG410 | 249 |
| MG420 | 241 |
| MG463 | 241 |

These results show a clear improvement of the present methods over current alignment techniques, since for each of the 35 successfully modeled sequences, the state-of-the-art, MODELLER program, failed. If these results are extrapolated to the entire Mycoplasma genitalium genome, the methods of the invention will allow approximately 40,000 residues to be accurately, structurally modeled, representing more than 30% of the soluble protein residues. Since the present methods are equally applicable to any genome, the present methods should offer similar modeling improvements across all genomes, including the human genome.

### EXAMPLE 5

Example 5 demonstrates that the methods of the invention provide superior three dimensional structures to the methods of R. Sánchez and A. \check{S}ali and the ModBASE for the first 180 sequences in the Mycoplasma genitalium genome. R. Sánchez and A. \check{S}ali, Bioinformatics, 15, 1060-1061 (1999). In this example, the three dimensional structures of the first 180 sequences in the Mycoplasma genitalium genome are determined using the preferred alignment techniques of the invention in combination with the three dimensional structure generating capabilities of MODELLER. The results of this experiment and the results of Sánchez and \check{S}ali are shown in Table 6. The first column in Table 6 shows the actual number of residues of each sequence. The remaining two columns show the number of residues that were correctly modeled by the methods according to the invention (3d column from the left) and the methods according to Sanchez and Sali (Far Right-hand Column). Substantially complete models containing at least 80% of the total sequence length are highlighted in bold. Structures generated by each method passed identical reliability tests. These tests are published (Sanchez and Sali 1998), and represent a threshold where the structures will have the correct fold with a confidence limit of >95%.

TABLE 6

| | #AA | B. | | Seq. | #AA | | |
|---|---|---|---|---|---|---|---|
| MG001 | 364 | 318 | 139 | MG084 | 290 | 107 | — |
| MG002 | 310 | 65 | — | MG088 | 155 | 140 | 137 |
| MG003 | 650 | — | 162 | MG089 | 688 | 171 | 679 |
| MG004 | 836 | 457 | 171 | MG090 | 208 | 94 | — |
| MG005 | 417 | 416 | 410 | MG091 | 160 | 99 | — |
| MG006 | 210 | 210 | — | MG093 | 150 | 146 | 144 |
| MG007 | 254 | 90 | — | MG094 | 446 | 337 | — |
| MG008 | 442 | 313 | — | MG097 | 245 | 227 | 227 |
| MG010 | 218 | 212 | — | MG098 | 477 | 86 | — |
| MG011 | 287 | 115 | — | MG099 | 477 | 190 | — |
| MG013 | 306 | 270 | — | MG102 | 315 | 307 | 294 |
| MG014 | 623 | 175 | — | MG104 | 725 | 120 | — |
| MG015 | 589 | 200 | — | MG105 | 200 | 139 | — |
| MG017 | 176 | 118 | — | MG106 | 226 | 186 | — |
| MG019 | 389 | 138 | 81 | MG107 | 189 | 184 | 182 |
| MG020 | 308 | 308 | 119 | MG108 | 260 | 260 | — |
| MG021 | 512 | 511 | — | MG109 | 362 | 288 | — |
| MG023 | 288 | 287 | 265 | MG111 | 433 | 433 | — |
| MG024 | 367 | 245 | — | MG112 | 209 | 206 | — |
| MG025 | 298 | 58 | — | MG113 | 456 | 453 | 435 |
| MG026 | 190 | 121 | — | MG116 | 251 | 96 | — |
| MG030 | 206 | 206 | 74 | MG118 | 340 | 340 | 321 |
| MG035 | 414 | 412 | 397 | MG119 | 564 | 419 | — |
| MG036 | 550 | 543 | — | MG122 | 709 | 571 | 599 |
| MG037 | 450 | 142 | — | MG123 | 471 | — | 159 |
| MG038 | 508 | 502 | 500 | MG124 | 102 | 102 | 92 |
| MG039 | 384 | 332 | 38 | MG125 | 285 | 277 | — |
| MG041 | 88 | 88 | 86 | MG126 | 347 | 341 | — |
| MG042 | 559 | 192 | — | MG127 | 145 | 134 | — |
| MG045 | 483 | 336 | — | MG128 | 259 | 63 | — |
| MG046 | 315 | 177 | — | MG129 | 117 | — | 68 |
| MG047 | 383 | 374 | 356 | MG132 | 141 | 109 | 101 |
| MG048 | 446 | 395 | 274 | MG136 | 490 | 484 | 482 |
| MG049 | 320 | 238 | 231 | MG137 | 404 | 84 | — |
| MG051 | 421 | 421 | 385 | MG138 | 598 | 285 | 475 |
| MG052 | 130 | 102 | 81 | MG140 | 1113 | — | 66 |
| MG053 | 550 | 521 | 406 | MG141 | 531 | 269 | — |
| MG057 | 178 | 82 | — | MG142 | 619 | 205 | 290 |
| MG058 | 297 | 286 | 41 | MG148 | 409 | 242 | — |
| MG060 | 297 | 120 | — | MG154 | 285 | 140 | — |
| MG062 | 680 | 148 | — | MG155 | 87 | 72 | — |
| MG063 | 255 | 252 | — | MG156 | 144 | 110 | — |
| MG065 | 466 | 212 | — | MG161 | 122 | 122 | 117 |
| MG066 | 648 | 622 | 628 | MG162 | 108 | 69 | — |
| MG068 | 474 | 52 | — | MG165 | 141 | 132 | 129 |
| MG069 | 908 | 243 | 234 | MG166 | 184 | 166 | — |
| MG070 | 284 | 167 | — | MG167 | 115 | 61 | — |
| MG072 | 806 | 124 | — | MG168 | 211 | 144 | 138 |
| MG073 | 656 | 599 | 89 | MG171 | 214 | 209 | 211 |
| MG077 | 407 | 76 | — | MG172 | 248 | 248 | 208 |
| MG079 | 402 | 93 | — | MG173 | 70 | 70 | 68 |
| MG080 | 848 | 104 | — | MG177 | 328 | 304 | 60 |
| MG081 | 137 | 128 | 74 | MG178 | 123 | 62 | — |
| MG082 | 226 | 221 | 216 | MG179 | 274 | 227 | — |
| MG083 | 189 | 185 | — | MG180 | 304 | 225 | — |

Probably, the single most important benchmark for determining the efficacy of an alignment method, is the ability of that method to be used to predict substantially complete structural models-i.e. correctly modeling at least 80% of residues correctly. The methods of the current invention modeled approximately 27% of the 180 Mycoplasma genitalitum sequences to least 80% accuracy, while ModBase only modeled 13% of the sequences to the same accuracy. Thus, the current alignment methods represent at least a two fold improvement over the current, state-of-the-art, alignment methods.

Another important standard for gauging the effectiveness of an alignment method, is the ability of that method to be used to predict the structure of complete domains correctly. Once again, when the methods of the current invention were used to construct three dimensional models, complete domains were accurately modeled for 106 of the 180 sequences (59%), versus only 48 of the 180 sequences (27%) in ModBase.

A third metric for measuring the effectiveness of an alignment method, is the ability of that method to be used to predict the three dimensional location of any one residue in a structural model. Again, when the methods of the current invention were used to construct three dimensional models, the coordinates of nearly 22,000 of the estimated 50,000 (or approximately 44%) soluble protein residues were accurately located, while ModBase faired less than half as well with approximately 21% of the residues properly located.

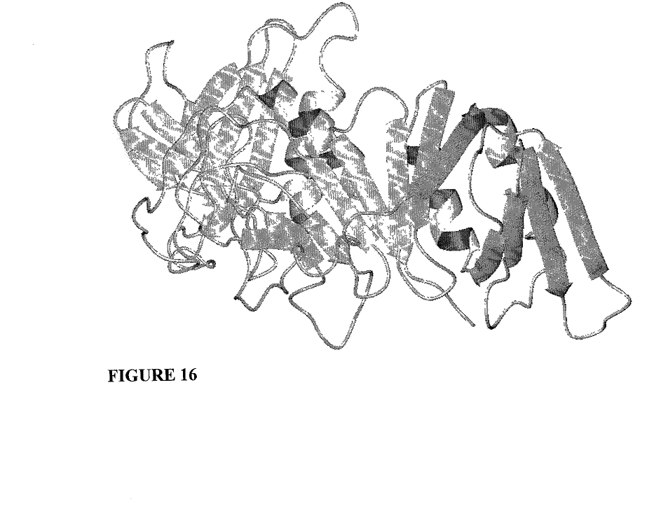

FIG. 16, shows a ribbon representation for MG001 based on the methods of the current invention used in combination with MODELLER. By contrast MODBASE only provides and incomplete, structural fragment, for the same sequence.

### EXAMPLE 6

Example 6 demonstrates that the methods of the invention, in combination with widely available homology modeling packages, may be used to predict accurate three dimensional structures at low sequence homologies. In this example consider the three dimensional structure of SC001 (orf YGL040C) from Brewer's yeast (Saccharomyces cerevisiae) is determined based upon a low homology template sequence. In order to build a BRIDGE/BULGE list, gapped-BLAST was used to determine a list of protein structures in the Protein Databank with similar sequences to the query sequence, SC001. The 8 PDB similar structures that were found are shown in Table 7.

TABLE 7

| | | | |
|---|---|---|---|
| 1ylvA | 1aw5 | 1b4eA | 1ylvA |
| 1aw5 | 1b4eA | 1b4kA | 1b4kB |

In order to further demonstrate the ability of the preferred alignment methods to generate accurate structures at low sequence homologies, the sequence 1b4kA (shown in Table 7) was used as a template sequence and to generate the BRIDGE/BULGE list. The structure alignment between SC001 and 1b4kA has a 35% sequence homology and a reliable structural model for sequence SC001 built from 1b4kA is not present in MODBASE. Structure 1b4kA is 326 residues long; there are 211 structurally aligned proteins in the FSSP file for 1b4kA. These alignments yield 3444 possible bridges and bulges for this structure, some of which are shown below in Table 8.

TABLE 8

| Template | Gap | Start Res. | End Res. | # Res. In |
|---|---|---|---|---|
| Protein | Type | In 1ovaA | In 1ovaA | Template |
| 1ovaC | BRIDGE | 341 | 354 | 1 |
| 1ovaB | BRIDGE | 65 | 79 | 1 |
| 1azxI | BULGE | 24 | 25 | 2 |
| 1azxI | BULGE | 62 | 63 | 3 |
| 1azxI | BRIDGE | 66 | 78 | 1 |
| 1azxI | BULGE | 92 | 94 | 1 |
| 1azxI | BRIDGE | 223 | 225 | 1 |
| 1azxI | BRIDGE | 269 | 272 | 1 |
| 1azxI | BULGE | 308 | 309 | 2 |
| 1azxI | BULGE | 316 | 317 | 3 |
| 1azxI | BULGE | 338 | 341 | 8 |
| 1azxI | BRIDGE | 345 | 348 | 2 |
| 1azxI | BRIDGE | 351 | 353 | 1 |
| 1by7A | BRIDGE | 63 | 65 | 1 |
| 1by7A | BRIDGE | 68 | 79 | 1 |
| 1by7A | BRIDGE | 91 | 98 | 1 |
| 1by7A | BRIDGE | 189 | 193 | 1 |
| 1by7A | BRIDGE | 235 | 237 | 1 |
| 1by7A | BULGE | 249 | 250 | 5 |
| 1by7A | BULGE | 308 | 309 | 2 |
| 1by7A | BRIDGE | 339 | 355 | 1 |

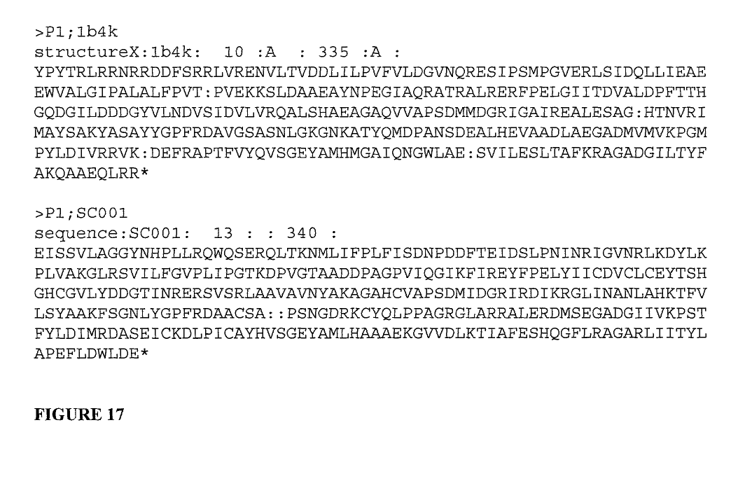

The optimal sequence alignment between SC001 to 1b4kA according to the methods of the invention is shown in PIR format in FIG. 17. The gap penalties used for this alignment were gap opening and extension penalties of 10.0 and 1.5, respectively, with bridge and bulge opening and extension penalties of 1.0 and 0.3, respectively. These gaps penalties were determined by optimizing the alignment obtained for sets of known structures.

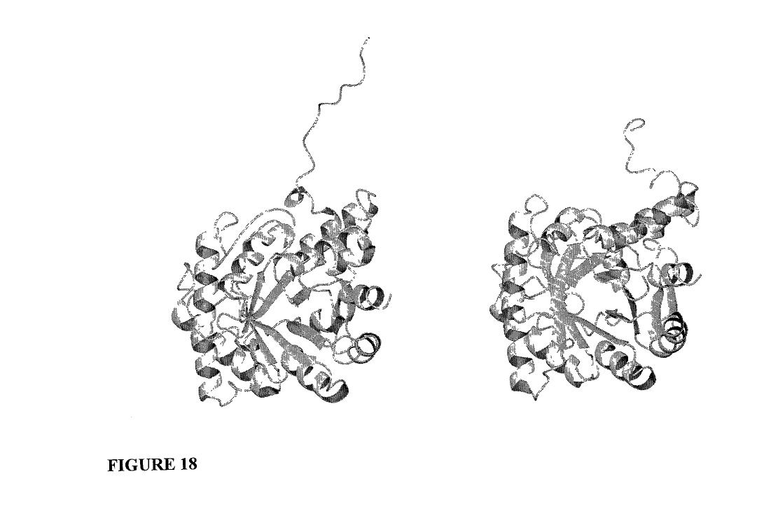

The PIR format alignment was then used as the alignment input for the MODELLER homology modeling software. The structure built by MODELLER using this alignment is compared to the actual crystal structure of SC001, law5, in FIG. 18 (law5 is on the left, prediction on the right). The alpha-carbon CRMS is 2.11 Å for 326 matched residues demonstrating that once again, the preferred alignment methods when used in combination with a homology modeling program were able to generate an accurate structural model when current methods failed.

### EXAMPLE 7

Example 7 demonstrates that the methods of the invention, in combination with widely available homology modeling packages, may be used to predict accurate three-dimensional structures at sequence homologies well below 25%.

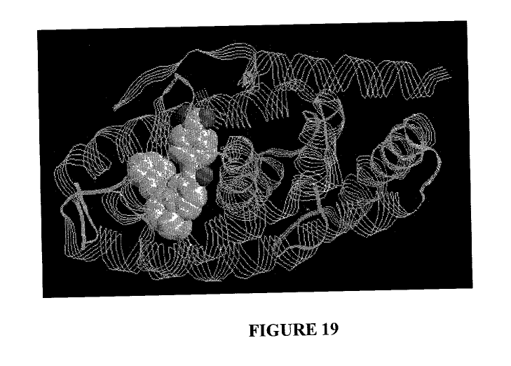

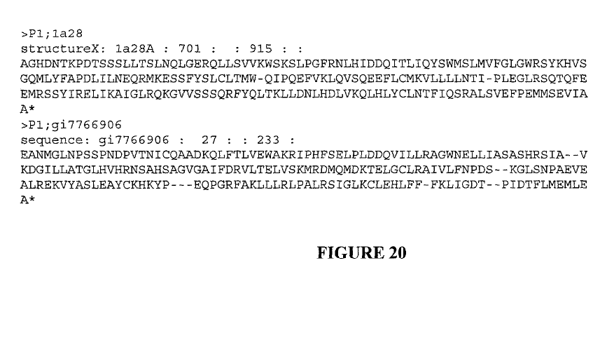

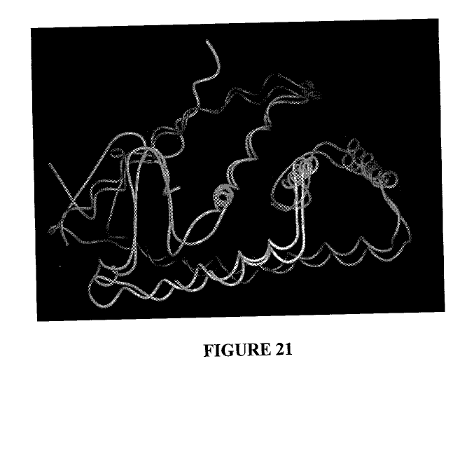

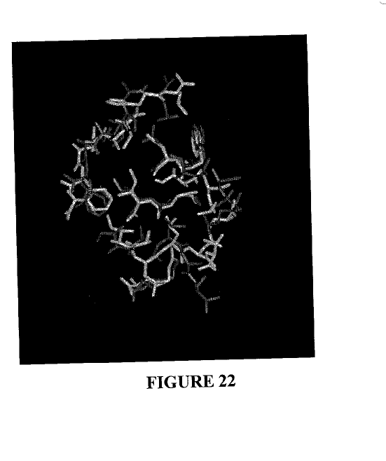

Consider the three dimensional structure of RXR retinoic acid receptor, chain A of PDB code 1 dkf. For this structure, the protein was co-crystallized with oleic acid. A ribbon diagram of the structure, showing the oleic acid ligand in space filling representation is shown in FIG. 19. FIG. 20 shows the STRUCTFAST alignment in PIR format between the sequence of 1dkf (denoted as gi7766906) and the sequence of chain A of structure 1a28, denoted 1a28A. In total, 197 residues are aligned to the template, and sequence identity is only 19%. FIG. 21 shows a rainbow ribbon overlay between the predicted structure and the crystal structure of chain A of 1dkf. The alpha-carbon CRMS for the best aligning 158 residues (80% of the complete 197 residues) is 1.6 Å. FIG. 22 shows an overlay of the predicted structure (darker) and crystal structure (lighter) for the 22 key residues that form the oleic acid binding pocket. The backbone atoms in these 22 residues overlay to 1.7 Å, and all of the heavy atoms in the residues, including the sidechain atoms, overlay to 2.2 Å.

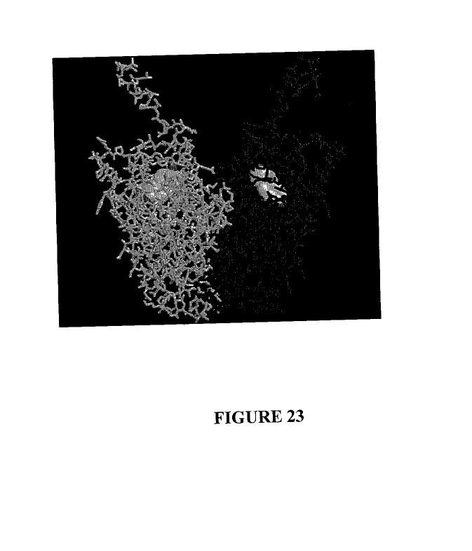

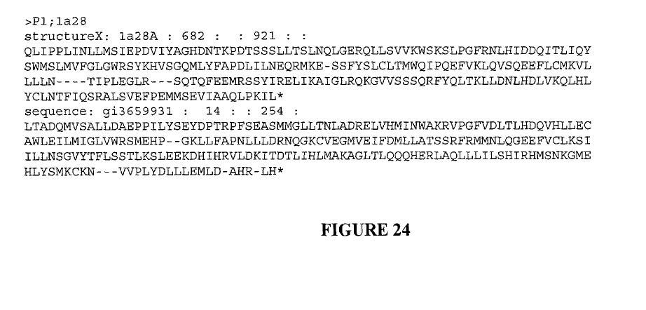

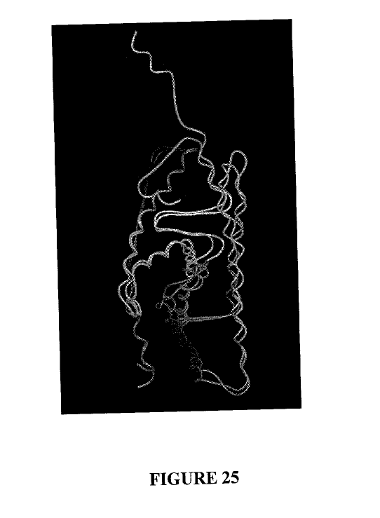

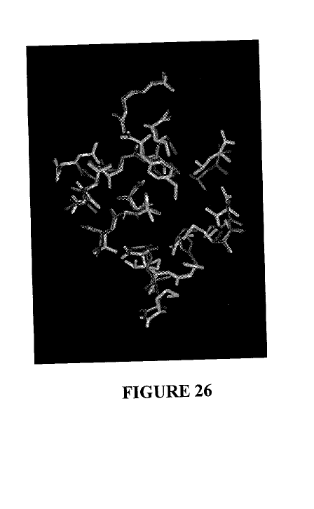

Consider the three dimensional structure of an estrogen receptor, chain A of PDB code 1a52. For this structure, the protein was co-crystallized as a dimer with estradiol. A stick diagram of the structure, showing the estradiol ligands in space filling representation is shown in FIG. 23. FIG. 24 shows the alignment according to the methods of the invention, in PIR format, between the sequence of the estrogen receptor (denoted as gi3659931) and the sequence of chain A of structure 1a28, denoted 1a28A. In total, 241 residues are aligned to the template, and sequence identity is 23%. FIG. 25 shows a rainbow ribbon overlay between the predicted structure according to the methods of the invention of the estrogen receptor and the crystal structure of chain A of 1a52. The alpha-carbon CRMS for the best aligning 193 residues (80% of the complete 241 residues) is 1.9 Å. FIG. 26 shows an overlay of the predicted structure (darker) and crystal structure (lighter) for the 19 key residues that form the estradiol binding pocket. The backbone atoms in these 19 residues overlay to 0.8 Å, and all of the heavy atoms in the residues, including the side-chain atoms, overlay to 1.8 Å.

### EXAMPLE 8

Example 8 demonstrates that the methods of the invention, in combination with widely available homology modeling packages, may be used to predict accurate three-dimensional structures of proteins located in the cell membrane at low sequence homology.

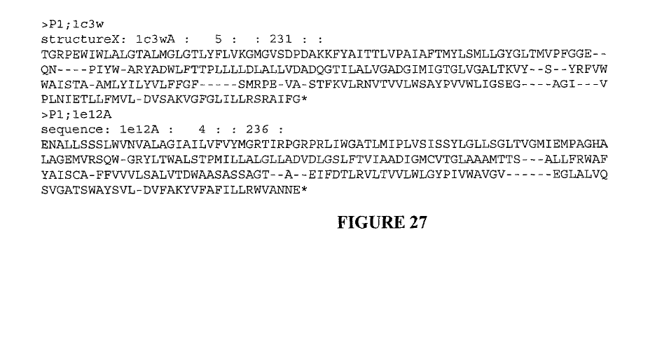

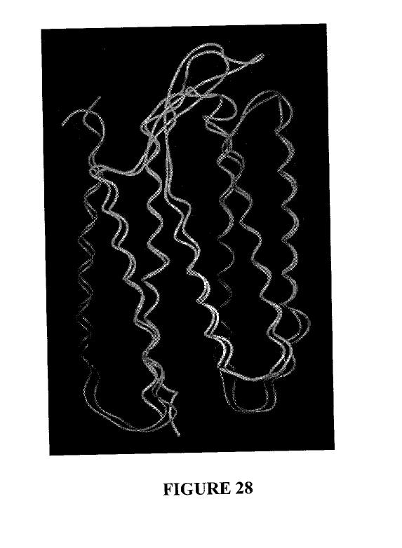

FIG. 27 shows the alignment, in PIR format, between the sequence of halorhodopsin, denoted 1e12A, and the sequence of bacteriorhodopsin, denoted 1c3wA made by the methods according to the invention. In total, 233 residues are aligned to the template, and the sequence identity is 32%. FIG. 28 shows a rainbow ribbon overlay between the three-dimensional structure created using the alignment in FIG. 27, compared to the halorhodopsin crystal structure, chain A of PDB code 1e12. The alpha-carbon CRMS for the best aligning 187 residues (80% of the complete 233 residues) is 0.91 Å.

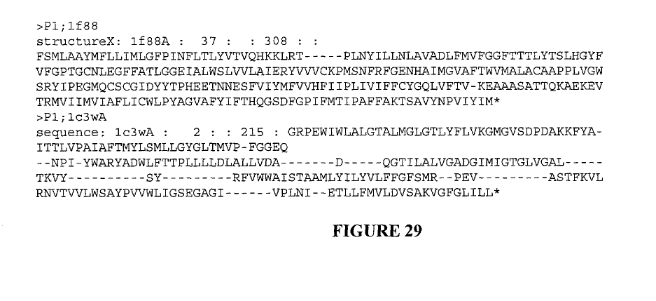

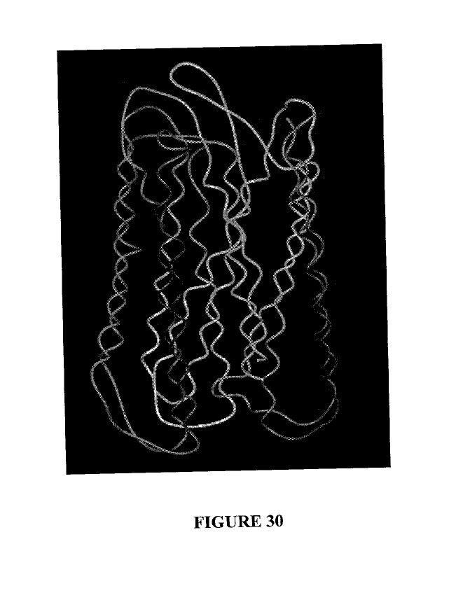

FIG. 29 shows the alignment formed from the methods according to the invention in PIR format, between the sequence of bacteriorhodopsin, denoted 1c3wA, and the sequence of rhodposin, chain A of PDB structure 1f88, denoted 1f88A. In total, 214 residues are aligned to the template, and the sequence identity is only 13%. FIG. 30 shows a rainbow ribbon overlay between the three-dimensional structure created using the alignment in FIG. 29, compared to the bacteriorhodopsin crystal structure, chain A of PDB code 1c3w. The alpha-carbon CRMS for the best aligning 172 residues (80% of the complete 214 residues) is 5.24 Å.

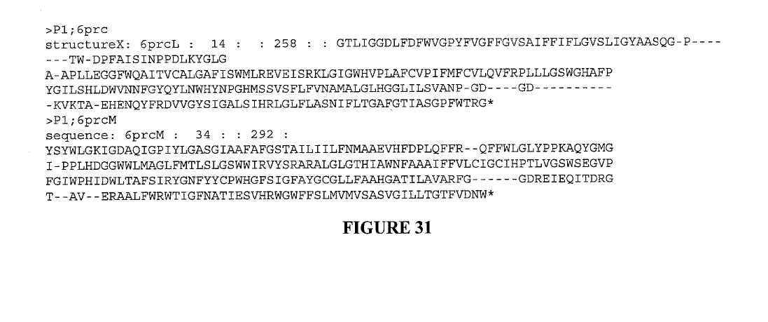

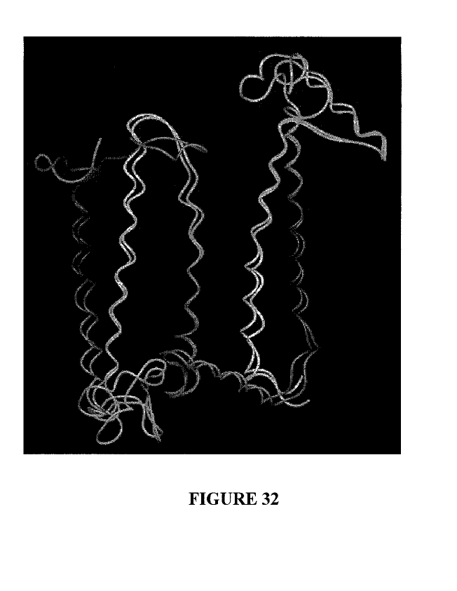

FIG. 31 shows the alignment, formed from the method according to the invention, in PIR format, between the sequence of a membrane spanning chain of the photosynthetic reaction center, denoted 6prcM, and the sequence of a different chain from the photosynthetic reaction center, chain L of PDB structure 6prc, denoted 6prcL. In total, 259 residues are aligned to the template, and the sequence identity is 28%. FIG. 32 shows a rainbow ribbon overlay between the three-dimensional structure created using the alignment in FIG. 31, compared to the crystal structure for chain M of PDB code 6prc. The alpha-carbon CRMS for the best aligning 207 residues (80% of the complete 259 residues) is 1.00 Å.

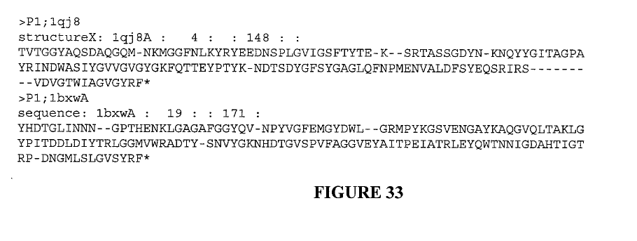

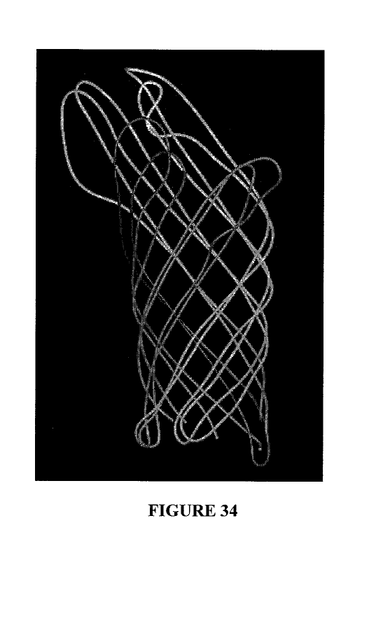

FIG. 33 shows the alignment, according to the methods of the invention, in PIR format, between the sequence of ompA, denoted 1bxwA, and the sequence of ompX, chain A of PDB structure 1qj8, denoted 1qj8A. In total, 153 residues are aligned to the template, and the sequence identity is only 21%. FIG. 34 shows a rainbow ribbon overlay between the three-dimensional structure created using the alignment in FIG. 33, compared to the ompA crystal structure, chain A of PDB code 1bxw. The alpha-carbon CRMS for the best aligning 172 residues (80% of the complete 214 residues) is 2.59 Å.

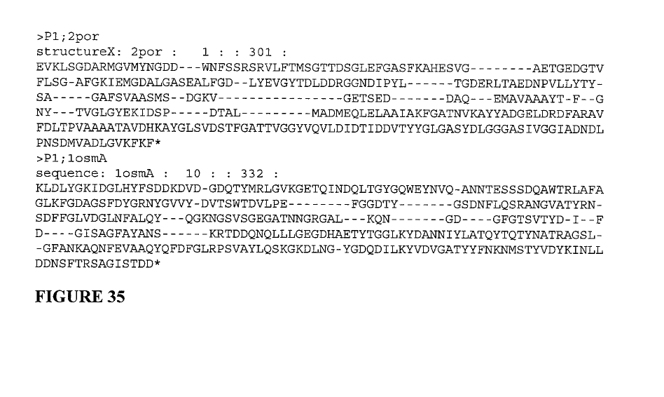

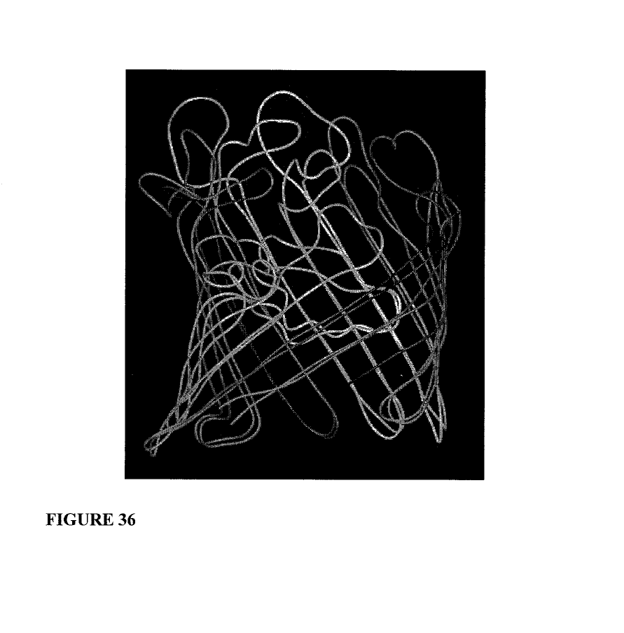

FIG. 35 shows the alignment, according to the methods of the invention, in PIR format, between the sequence of ompK36, denoted 1osmA, and the sequence of porin protein 2por. In total, 323 residues are aligned to the template, and the sequence identity is only 12%. FIG. 36 shows a rainbow ribbon overlay between the three-dimensional structure created using the alignment in FIG. 35, compared to the ompK36 crystal structure, chain A of PDB code 1osm. The alpha-carbon CRMS for the best aligning 259 residues (80% of the complete 323 residues) is 3.11 Å.

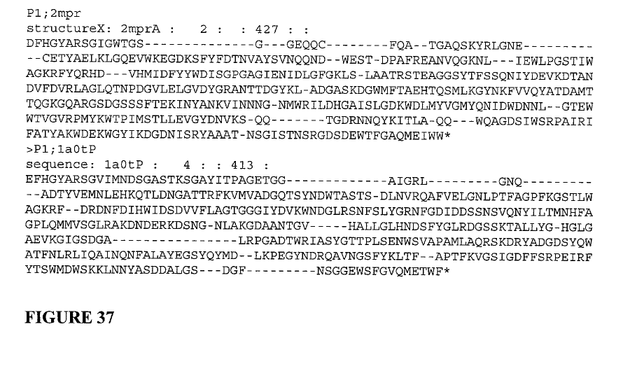

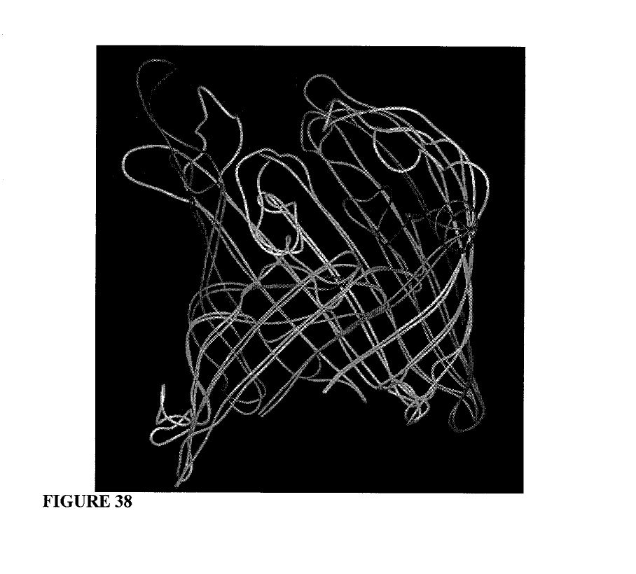

FIG. 37 shows the alignment, formed from the methods according to the invention, in PIR format, between the sequence of sucrose-specific porin, denoted 1a0tP, and the sequence of maltoporin, chain A of PDB structure 2mpr, denoted 2mprA. In total, 410 residues are aligned to the template, and the sequence identity is 21%. FIG. 38 shows a rainbow ribbon overlay between the three-dimensional structure created using the alignment in FIG. 37, compared to the sucrose-specific porin crystal structure, chain P of PDB code 1a0tP. The alpha-carbon CRMS for the best aligning 328 residues (80% of the complete 410 residues) is 2.26 Å.

Although the invention has been described with reference to preferred embodiments and specific examples, it will be readily appreciated by those skilled in the art that many modifications and adaptations of the invention are possible without deviating from the spirit and scope of the invention. Thus, it is to be clearly understood that this description is made only by way of example and not as a limitation on the scope of the invention as claimed below.

What is claimed is:

1. A method for determining a preferred alignment between a query sequence and at least one template sequence comprising the steps of:
a. aligning at least two reference sequences to determine one or more BRIDGE/BULGE gaps;
b. determining at least one alignment score between the potential alignment of said query sequence and each said template sequence; wherein each said alignment score reflects whether said alignment between said query sequence and each said template sequence creates a BRIDGE/BULGE gap; and
c. determining a preferred alignment between said query sequence and each said template sequence based on said alignment score.

2. The method of claim 1 wherein said preferred alignment is the optimal alignment.

3. The method of claim 1 wherein step b comprises the steps of:
a. forming a sequence alignment similarity matrix for said query sequence and each said template sequence with matrix elements sᵢⱼ; and
b. determining a sequence alignment sum matrix with matrix elements Sᵢⱼ from the dynamic evolution of each said sequence alignment similarity matrix, wherein the matrix elements of each said sum matrix reflect whether or not any potential alignment gaps that may be formed from the alignment of said query sequence with each said template sequence creates a BRIDGE/BULGE gap.

4. The method of claim 3 wherein step b comprises the steps of:
a. calculating said sequence alignment sum matrix from the dynamic evolution of each said sequence alignment similarity matrix, according to the equation:
Sᵢⱼ=sᵢⱼ+Max{S_{i+1,j+1}, Si_{+1,j+2 to jmax}−GAP, S_{i+2 to imax,j+2}−GAP, S_{m,n}−BRIDGE/BULGE},
wherein GAP represents the gap penalty for an alignment gap between said query sequence and each said template sequence, BRIDGE/BULGE represents the penalty for a known bridge or bulge that begins at the m, n matrix element of said sum matrix and ends at the i, j matrix element of said sum matrix and Max{S_{i+1,j+1}, Si_{+1,j+2 to jmax}−GAP, S_{i+2 to imax, j+2}−GAP, S_{m,n}−BRIDGE/BULGE} refers to the maximum value of the four terms contained within the brackets

5. A method for determining a preferred alignment between a query sequence and at least one template sequence comprising the steps of:
a. aligning at least two reference sequences to determine at least one BRIDGE/BULGE gap;
b. forming a sequence alignment similarity matrix from said query sequence and each said template sequence;
c. determining a sequence alignment sum matrix from the dynamic evolution of each said sequence alignment similarity matrix, wherein the matrix elements of each said sum matrix reflect whether or not any potential alignment gaps that may be formed from the alignment of said query sequence with each said template sequence creates a BRIDGE/BULGE gap; and
d. determining a preferred alignment between said query sequence and each said template sequence from said dynamic evolution of each said sum matrix.

6. The method of claim 5 wherein said preferred alignment is the optimal alignment.

7. A method for determining a preferred alignment between a query sequence and at least one template sequence comprising the steps of:
a. aligning at least two reference sequences to determine at least one BRIDGE/BULGE gap;
b. calculating a sequence alignment similarity matrix with matrix elements sᵢⱼ for said query sequence and each said template sequence;
c. calculating a sequence alignment sum matrix with matrix elements Sᵢⱼ from the dynamic evolution of each said sequence alignment similarity matrix, according to the equation:
Sᵢⱼ=sᵢⱼ+Max{S_{i+1,j+1}, Si_{+1,j+2 to jmax}−GAP, S_{i+2 to imax,j+2}−GAP, S_{m,n}−BRIDGE/BULGE},
wherein GAP represents the gap penalty for an alignment gap between said query sequence and each said template sequence, BRIDGE/BULGE represents the penalty for a known bridge or bulge that begins at the m, n matrix element of said sum matrix and ends at the i, j matrix element of said sum matrix and Max{S_{i+1,j+1}, S_{i+1,j+2 to jmax}−GAP, S_{i+2 to imax, j+2}−GAP, S_{m,n}−BRIDGE/BULGE} refers to the maximum value of the four terms contained within the brackets; and
d. determining a preferred alignment between said query sequence and each said template sequence from the dynamic evolution of said sum matrix.

8. The method of claim 7 wherein said preferred alignment is an optimal alignment.

9. A method for determining a preferred alignment between at least one query sequence and at least one template sequence, for use in primary sequence homology modeling methods, comprising the steps of:
a. aligning at least two reference sequences to determine one or more BRIDGE/BULGE gaps;
b. determining at least one alignment score for the potential alignment of each said query sequence and each said template sequence; wherein each said alignment score reflects whether said alignment between each aid query sequence and each said template sequence creates a BRIDGE/BULGE gap; and
c. determining a preferred alignment between each said query sequence and each said template sequence based on said alignment scores, wherein said preferred alignment contains from approximately 10% to approximately 20% homologous residues.

10. The method of claim 9 wherein said preferred alignment is the optimal alignment.

11. The method of claim 9 wherein said primary sequence homology method is a method for determining the three dimensional structure of said query sequence.

12. The method of claim 10 wherein said primary sequence homology modeling method is a method for determining the three dimensional structure of said query sequence.

13. The method of claim 9 wherein said primary sequence homology modeling method is a method for determining the primary sequence homology relationship between at least two query sequences.

14. The method of claim 10 wherein said primary sequence homology modeling method is a method for determining the sequence homology relationship between at least two query sequences.

15. A method for determining a preferred alignment between at least one query sequence and at least one template sequence, for use in primary sequence homology modeling methods, comprising the steps of:
a. aligning at least two reference sequences to determine at least one BRIDGE/BULGE gap;
b. forming a sequence alignment similarity matrix for each said query sequence and each said template sequence;
c. determining a sequence alignment sum matrix from the dynamic evolution of each said sequence alignment similarity matrix, wherein the matrix elements of each said sum matrix reflect whether or not any potential alignment gaps that may be formed from the alignment of each said query sequence with each said template sequence creates a BRIDGE/BULGE gap; and
d. determining a preferred alignment between each said query sequence and each said template sequence from said dynamic evolution of each said sum matrix, wherein said preferred alignment contains from approximately 10% to approximately 20% homologous residues.

16. The method of claim 15 wherein said preferred alignment is the optimal alignment.

17. The method of claim 15 wherein said primary sequence homology method is a method for determining the three dimensional structure of said query sequence.

18. The method of claim 16 wherein said primary sequence homology modeling method is a method for determining the three dimensional structure of said query sequence.

19. The method of claim 15 wherein said primary sequence homology modeling method is a method for determining the primary sequence homology relationship between at least two query sequences.

20. The method of claim 16 wherein said primary sequence homology modeling method is a method for determining the primary sequence homology relationship between at least two query sequences.

21. A method for determining a preferred alignment between at least one query sequence and at least one template sequence, for use in primary sequence homology modeling methods, comprising the steps of:
a. aligning at least two reference sequences to determine at least one BRIDGE/BULGE gap;
b. calculating a sequence alignment similarity matrix with matrix elements Sᵢⱼ for each said query sequence and each said template sequence;
c. calculating a sequence alignment sum matrix with matrix elements Sᵢⱼ from the dynamic evolution of each said sequence alignment similarity matrix, according to the equation:
Sᵢⱼ=sᵢⱼ+Max{S_{i+1,j+1}, Si_{+1,j+2 to jmax}−GAP, S_{i+2 to imax, j+2}−GAP, S_{m, n}−BRIDGE/BULGE},
wherein GAP represents the gap penalty for an alignment gap between said query sequence and each said template sequence, BRIDGE/BULGE represents the penalty for a known bridge or bulge that begins at the m, n matrix element of said sum matrix and ends at the i, j matrix element of said sum matrix and Max{S_{i+1,j+1}, Si_{+1,j+2 to jmax}−GAP, S_{i+2 to imax, j+2}−GAP, S_{m,n}−BRIDGE/BULGE} refers to the maximum value of the four terms contained within the brackets; and
d. determining a preferred alignment between each said query sequence and each said template sequence from the dynamic evolution of said sum matrix; wherein said preferred alignment contains from approximately 10% to approximately 20% homologous residues.

22. The method of claim 21 wherein said preferred alignment is the optimal alignment.

23. The method of claim 21 wherein said primary sequence homology modeling method is a method for determining the three dimensional structure of said query sequence.

24. The method of claim 22 wherein said primary sequence homology modeling method is a method for determining the three dimensional structure of said query sequence.

25. The method of claim 21 wherein said primary sequence homology modeling method is a method for determining the primary sequence homology relationship between at least two query sequences.

26. The method of claim 22 wherein said primary sequence homology modeling method is a method for determining the primary sequence homology relationship between at least two query sequences.

27. A method for determining the three dimensional structure of a query sequence based upon primary sequence homology modeling with at least one template sequence, wherein, said alignment between said query sequence and said template sequence is determined by the methods of claim 2, claim 6, claim 8, claim 12, claim 20 and claim 24.

28. A method for determining the primary sequence homology relationship between at least two query sequences based upon primary sequence homology modeling with at least one template sequence, wherein, said alignment between said query sequence and said template sequence is determined by the methods of claim 2, claim 6, claim 8, claim 14, claim 22, and claim 26.
